(19) United States
(12) Patent Application Publication
(10) Pub. No.: US 2026/0232854 A1
(43) Pub. Date: Aug. 13, 2026

(54) GERMICIDAL APPARATUS FOR PROVIDING UV RADIATION

(71) Applicant: HS Ultra LLC, Cameron, WI (US)

(72) Inventors: Alex Haag, Cameron, WI (US); Brent Spurling, Luck, WI (US)

(21) Appl. No.: 19/127,308

(22) PCT Filed: Nov. 3, 2023
(86) PCT No.: PCT/US2023/078592

§ 371 Date: May 5, 2025

Related U.S. Application Data
(60) Provisional application No. 63/382,797, filed on Nov. 8, 2022.

(51) Int. Cl. A61L 2/10; A61L 2/24

(52) U.S. Cl. CPC A61L 2/10; A61L 2/24; A61L 2202/16

(57) ABSTRACT

Systems and methods for providing an apparatus for distribution ultraviolet (UV) radiation are provided herein. For example, an apparatus including a light-source housing assembly is provided. The light-source housing assembly may include a first light-source holder configured to hold a first end of at least one UV bulb and a second light-source holder configured to hold a second end of the at least one UV bulb. The light-source housing assembly may optionally include a directionalization cover extending from the first end to the second end of the light-source housing assembly, and a transmittance cover extending from the first end to the second end of the light-source housing assembly. The apparatus may also include an arm having a distal end and a proximal end, and an attachment component that is moveably coupled to the proximal end of the arm and moveably coupled to the first end of the light-source housing assembly.

### CROSS REFERENCE TO RELATED APPLICATION(S)

This application is a 371 National Stage filing claiming priority to and benefit of PCT Application No.: PCT/US2023/078592, filed Nov. 3, 2023; which claims the benefit of and priority to U.S. Provisional patent application Ser. No. 63/382,797, filed on Nov. 8, 2022, all of which are hereby incorporated by reference herein.

### FIELD

Embodiments of the present disclosure are directed in general to the field of disinfection of microbes, and in particular to the distribution of ultra-violet (UV) radiation for disinfection purposes.

### BACKGROUND

UV radiation can be used for disinfection purposes by inactivating microorganisms on surfaces as well as in the air and in liquids. Generally, UV radiation devices, when deployed, emit UV light, particularly UVC light, that can be absorbed by microorganisms and damage nucleic acids and proteins therein, thereby leading to inactivation (i.e., killing) of the microorganism. Specifically, UV radiation devices emit UVC light having a wavelength between 100-280 nm, which is the most effective part of the UV spectrum for germicidal disinfection.

UV radiation can provide various advantages over other disinfection techniques. For example, UV radiation can be performed automatically, remotely, and used to disinfect a variety of environments, such as surfaces, liquids, and air. Moreover, UV radiation does not involve any chemicals or specialized materials, and thus can be used to disinfect without contaminating an area with adverse chemicals or impacting the disinfection zone beyond abating undesirable microorganisms. For example, UV radiation is often used in hospitals for disinfection purposes because the use of chemicals may be irritating or harmful to patients and staff. UV radiation is also used to disinfect food products, where ingestion of disinfecting chemicals could be harmful to a consumer. Additionally, disinfection via UV radiation is often time and cost effective because it generally involves a single step of emitting UVC light onto the disinfection zone.

### BRIEF SUMMARY

In one aspect, an apparatus for distributing UV radiation is provided. The apparatus for distribution ultraviolet (UV) radiation may include a light-source housing assembly having a first end and a second end. The light-source housing assembly may include a first light-source holder configured to hold a first end of at least one UV bulb within the light-source housing assembly and second light-source holder configured to hold a second end of the at least one UV bulb within the light-source housing assembly. The first light-source holder is positioned proximate to the first end of the light-source housing assembly and the second light-source holder may be positioned proximate to the second end of the light-source housing assembly. Optionally, the at least one UV bulb is optionally included in the light-source housing assembly. The light-source housing assembly may also include a directionalization cover extending from the first end of the light-source housing assembly to the second end of the light-source housing assembly and a transmittance cover extending from the first end of the light-source housing assembly to the second end of the light-source housing assembly. The directionalization cover may include at least one surface for directing emittance of UV light from the at least one UV bulb. Optionally, the at least one surface of the directionalization cover may include a first surface, a second surface, and a third surface, where the first surface is oriented to direct emittance of UV light in a first direction, the second surface is oriented to direct emittance of the UV light in a second direction, and the third surface is oriented to direct emittance of the UV light in a third direction. The first direction, second direction, and third direction may be different directions from each other. The transmittance cover may transmit the UV light from the at least one UV bulb.

The apparatus may also include a power source configured to provide power to the at least one UV bulb of the light-source housing assembly, an arm having a distal end and a proximal end, and an attachment component. For example, the power source may include one or more of a battery, an outlet plug, or a motorized vehicle. The arm may include a moveable element. In some embodiments, the arm may include a first segment and a second segment, and the moveable element may form a telescoping arm with the first segment and the second segment. In other embodiments, the arm may include a first segment and a second segment. The second segment may be coupled to the attachment component at the proximal end of the arm and may be independently rotatable from the first segment. Optionally, the second segment may be rotatably independent from the first segment such that the first segment rotates in a first direction that is transverse to a longitudinal axis of the second segment.

The attachment component may be moveably coupled to the proximal end of the arm and moveably coupled to the first end of the light-source housing assembly. For example, the attachment component may be moveably coupled to the light-source housing assembly by a pivoting component allowing for at least 180° rotation or at least 360° rotation of the light-source housing assembly with respect to a longitudinal axis of the arm. Optionally, the attachment component may provide 6 degrees of freedom for the light-source housing assembly with respect to the arm.

In some embodiments, the attachment component may include a pivotable connection and a stub arm having a first end and a second end. The pivotable connection may be coupled to the second end of the stub arm to the proximal end of the arm and the first end of the stub arm may be coupled to the first end of the light-source housing assembly. The pivotable connection may allow the stub arm to be rotatably independent with respect to the arm, allowing for at least 180° rotation of the stub arm with respect to the arm.

In some embodiments, the light-source housing assembly may be configured to hold two UV bulbs. In such cases, the first light-source holder may be configured to hold the first end of a first UV bulb and a first end of a second UV bulb, and the second light-source holder may be configured to hold the second end of the first UV bulb and a second end of the second UV bulb. In some embodiments, the apparatus may be a handheld device. In other embodiments, the apparatus may include a second attachment component that is configured to couple to the distal end of the arm and configured to couple the arm with a motorized vehicle. Optionally, the second attachment component may removably couple the distal end of the arm with the motorized vehicle.

In some embodiments, the apparatus may include a computing system and one or more sensors for providing positional feedback on a position of the light-source housing assembly to the computing assembly. Optionally, the one or more sensors may be configured to provide feedback of a disinfection zone achieved by the light-source housing assembly to the computing system.

In some embodiments, the apparatus may further include a second light-source housing assembly having a first end and a second end. The second light-source housing assembly may include a third light-source holder configured to hold a first end of at least one UV bulb within the second light-source housing assembly, and a fourth light-source holder configured to hold a second end of the at least one UV bulb within the second light-source housing assembly. The second light-source housing assembly may also include a second directionalization cover extending from the first end of the second light-source housing assembly to the second end of the second light-source housing assembly, where the directionalization cover includes at least one surface for directing emittance of UV light from the at least one UV bulb, and a second transmittance cover extending from the first end of the second light-source housing assembly to the second end of the second light-source housing assembly, where the transmittance cover transmits the UV light from the at least one UV bulb. In such cases, the power source may be further configured to provide power to the at least one UV bulb of the second light-source housing assembly.

In embodiments including the second light-source housing assembly, the apparatus may further include a pivotable connection and a first stub arm having a first end and a second end. The pivotable connection may couple the second end of the first stub arm to the proximal end of the arm and the first end of the first stub arm may be coupled to the first end of the light-source housing assembly. In some embodiments, the apparatus may further include a second stub arm having a first end and a second end, where pivotable connection couples the second end of the second stub arm to the proximal end of the arm and the first end of the second stub arm is coupled to the first end of the second light-source housing assembly. The pivotable connection may allow the first stub arm to be rotatably independent with respect to the arm and the second stub arm to be rotatably independent with respect to the arm.

In another aspect, a method for disinfecting a surface using UV radiation may be provided. The method may include providing an apparatus for distributing UV radiation. The apparatus may include a light-source housing assembly having a first end and a second end. The light-source housing assembly may include at least one UV bulb operable to emit UV radiation, a first light-source holder configured to hold a first end of the at least one UV bulb within the light-source housing assembly, where the first light-source holder is positioned proximate to the first end of the light-source housing assembly, and a second light-source holder configured to hold a second end of the at least one UV bulb within the light-source housing assembly, where the second light-source holder is positioned proximate to the second end of the light-source housing assembly. The apparatus may also include a directionalization cover extending from the first end of the light-source housing assembly to the second end of the light-source housing assembly, where the directionalization cover comprises at least one surface for directing emittance of UV light from the at least one UV bulb, and a transmittance cover extending from the first end of the light-source housing assembly to the second end of the light-source housing assembly, where the transmittance cover transmits the UV light from the at least one UV bulb. The apparatus may also include a power source configured to provide power to the at least one UV bulb of the light-source housing assembly, an arm having a distal end and a proximal end, where the arm comprises a moveable element, and an attachment component that can be moveably coupled to the proximal end of the arm and moveably coupled to the first end of the light-source housing assembly.

The method may also include positioning the light-source housing assembly in a first position relative to the arm. For example, positioning the light-source housing assembly in the first position relative to the arm may include directionalizing the UV radiation from the at least one UV bulb towards a first disinfection zone. The method may also include distributing UV radiation using the light-source housing assembly in the first position. For example, distributing UV radiation using the light-source assembly in the first position may include providing a UV radiation dosage that is greater than 10,000 μW/cm² at a distance of 10 inches or less from a disinfection zone, such as the first disinfection zone.

The method may also include positioning the light-source housing assembly in a second position relative to the arm. For example, positioning the light-source assembly in the second position relative to the arm includes directionalizing the UV radiation from the at least one UV bulb towards a second disinfection zone, where the second disinfection zone is different than the first disinfection zone. The method may also include distributing UV radiation using the light-source housing assembly in the second position.

The above described and many other features and attendant advantages of embodiments of the present disclosure will become apparent and further understood by reference to the following detailed description when considered in conjunction with the accompanying drawings.

### BRIEF DESCRIPTION OF THE DRAWINGS

These FIGURES are intended to be illustrative, not limiting. Although the Examples of the disclosure are generally described in the context of these FIGURES, it should be understood that it is not intended to limit the scope of the disclosure to these particular Examples.

FIG. 1 illustrates an example apparatus for distributing UV radiation, according to an embodiment herein;

FIG. 2 provides another view of the example apparatus for distributing UV radiation of FIG. 1, according to an embodiment herein;

FIGS. 3A-3C illustrate example cross-sectional views of a light-source housing assembly, according to an embodiment herein;

FIG. 4 illustrates an example apparatus for distributing UV radiation having two light-source housing assemblies, according to an embodiment herein;

FIG. 5 illustrates another example apparatus for distributing UV radiation, according to an embodiment herein;

FIG. 6 illustrates another example apparatus for distributing UV radiation having two light-source housing assemblies, according to an embodiment herein;

FIG. 7 illustrates example use-configurations for disinfecting surfaces using an example apparatus for distributing UV radiation, according to an embodiment herein;

FIGS. 8A and 8B illustrate example use-configuration for using apparatuses for distributing UV radiation in combination with a motorized vehicle, according to an embodiment herein;

FIG. 9 illustrates another example use-configuration for using an apparatus for distributing UV radiation in combination with a motorized vehicle, according to an embodiment herein;

FIG. 10 illustrates an example use-configuration of disinfecting surfaces using an example apparatus for distributing UV radiation in combination with a motorized vehicle, according to an embodiment herein;

FIG. 11 shows an example method for disinfecting a space using an apparatus for distributing UV radiation, according to an embodiment herein; and

FIG. 12 shows an example computing device suitable for distributing UV radiation using the systems and apparatuses provided herein, according to an embodiment herein.

### DETAILED DESCRIPTION

Examples are described herein in the context of systems, apparatuses, and methods for distributing UV radiation for disinfection purposes. Those of ordinary skill in the art will realize that the following description is illustrative only and is not intended to be in any way limiting. Reference will now be made in detail to implementations of examples as illustrated in the accompanying drawings. The same or similar reference indicators (e.g., 105 may indicate the same or similar component as 205) will be used throughout the drawings and the following description to refer to the same or like items.

In the interest of clarity, not all of the routine features of the examples described herein are shown and described. It will, of course, be appreciated that in the development of any such actual implementation, numerous implementation-specific decisions must be made in order to achieve the developer's specific goals, such as compliance with application- and business-related constraints, and that these specific goals will vary from one implementation to another and from one developer to another.

Recent events, such as the SARS-CoV-2 pandemic, have highlighted the need for improved disinfection or sterilization applications. A common disinfection application is UV disinfection or ultraviolet germicidal irradiation (UVGI). UV disinfection uses UV light, specifically UVC light, to break down certain chemical bonds and disrupt the structure of DNA, RNA, and proteins within a microorganism. These disruptions can cause the microorganism to be unable to multiply. Since the microorganism cannot multiply, the microorganism is considered to be inactivated as it cannot reproduce within a host and thus is no longer infectious.

UV radiation is effective for disinfection of surfaces by inactivation of both viruses and bacteria, though some organisms are more susceptible to UV inactivation than others. For example, because microorganisms come in a variety of sizes and shapes that affect their UV absorption, the effects of UV radiation can vary depending on the species of microorganism. Every microorganism, based on its biological make-up, has a unique spectral sensitivity. In other words, depending on the UVC wavelength range being used, each microorganism will require a different amount of energy to be inactivated. UV radiation dosage can be determined based on the intensity of the UVC energy and the exposure time at a specific wavelength.

For surface disinfection applications, the UV dose is the product of the irradiance (UV intensity) and exposure time:

$$Dose ( \frac{mJ}{c {m}^{2}} ) = Irradiance ( \frac{m W}{c {m}^{2}} ) * Exposure Time ( s )$$

Where: UV dose is measured in joules per meter squared (J/m²) or millijoules per centimeter squared (mJ/cm²); UV Intensity (also called UV irradiance) is measured in milliwatts per centimeter squared (mW/cm²); and exposure time is measured in seconds.

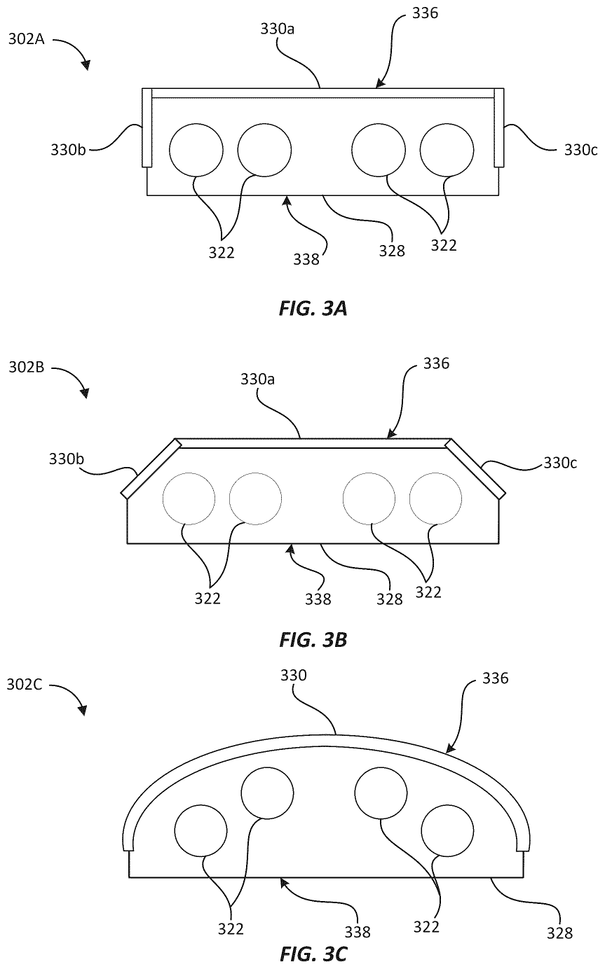

The predictable amount of dosage required for a specific degree of disinfection is referred to as a “log reduction” (i.e., logarithmic reduction). Log reduction relates to the percentage of microorganisms physically removed or inactivated by a given process. For example, a 1 log reduction will see the pathogen of interest reduced by 90% from the influent level before UV disinfection. The microbe count is reduced by a factor of 10- or 1 log. Thus, a 2 log reduction will see a 99% reduction, or microbe reduction by a factor of 100, and so on and so forth. FIG. 3 shows the chart of log reduction. By determining the doses of the targeted microorganisms and pairing them with the desired log reduction, an effective disinfection system can be created for many disinfection applications. These desired log reductions can be seen in the appendix referenced earlier.

For surface disinfection applications, the UV dose is the product of the irradiance (UV intensity) and exposure time: The UV dose-response relationship determines what proportion of a specific microorganism is destroyed after a particular dose of UV radiation. This figure can be expressed as either the proportion of microorganisms inactivated or the proportion remaining as a function of UV dose. The UV dose-response is calculated using the following equation:

$$Log intactivation = log 10 ( \frac{No}{N} ) ere : No = concentration of infectious microorganisms before exposure to UV light , and N = c oncentration of infectious microorganisms after exposure to UV light . Each microorganism has a unique dose - response c urve .$$

As illustrated below by Table 1, different types of bacteria and viruses require different amounts of UV radiation (measured as micoWatts per square centimeter (μW/cm²)) to achieve a 99% reduction (2 Log reduction) of that specific microorganism (kill factor).

TABLE 1
UV Radiation Dosage Requirements to Achieve 90%
and 99% Reduction in Active Microorganisms

| | Energy Dosage of Ultraviolet radiation (UV dose) in | |
|---|---|---|
| | μWs/cm² needed for kill factor | |
| Microorganism: | 90% | 99% |
| Bacteria | (1 log reduction) | (2 log reduction) |
| Bacillus anthracis - Anthrax | 4,520 | 8,700 |
| Bacillus anthracis spores - Anthrax | 24,320 | 46,200 |
| spores | | |
| Bacillus magaterium sp. (spores) | 2,730 | 5,200 |
| Bacillus magaterium sp. (veg.) | 1,300 | 2,500 |
| Bacillus paratyphusus | 3,200 | 6,100 |
| Bacillus subtilis spores | 11,600 | 22,000 |
| Bacillus subtilis | 5,800 | 11,000 |
| Clostridium tetani | 13,000 | 22,000 |
| Corynebacterium diphtheriae | 3,370 | 6,510 |
| Ebertelia typhosa | 2,140 | 4,100 |
| Escherichia coli | 3,000 | 6,600 |
| Leptospiracanicola - infectious | 3,150 | 6,000 |
| Jaundice | | |
| Microccocus candidus | 6,050 | 12,300 |
| Microccocus sphaeroides | 1,000 | 15,400 |
| Mycobacterium tuberculosis | 6,200 | 10,000 |
| Neisseria catarrhalis | 4,400 | 8,500 |
| Phytomonas tumefaciens | 4,400 | 8,000 |
| Proteus vulgaris | 3,000 | 6,600 |
| Pseudomonas aeruginosa | 5,500 | 10,500 |
| Pseudomonas fluorescens | 3,500 | 6,600 |
| Salmonella enteritidis | 4,000 | 7,600 |
| Salmonela paratyphi - Enteric fever | 3,200 | 6,100 |
| Salmonella typhosa - Typhoid fever | 2,150 | 4,100 |
| Salmonella typhimurium | 8,000 | 15,200 |
| Sarcina lutea | 19,700 | 26,400 |
| Serratia marcescens | 2,420 | 6,160 |
| Shigella dyseteriae - Dysentery | 2,200 | 4,200 |
| Shigella flexneri - Dysentery | 1,700 | 3,400 |
| Shigella paradysenteriae | 1,680 | 3,400 |
| Spirillum rubrum | 4,400 | 6,160 |
| Staphylococcus albus | 1,840 | 5,720 |
| Staphylococcus aureus | 2,600 | 6,600 |
| Staphylococcus hemolyticus | 2,160 | 5,500 |
| Staphylococcus lactis | 6,150 | 8,800 |
| Streptococcus viridans | 2,000 | 3,800 |
| Vibrio comma - Cholera | 3,375 | 6,500 |
| Molds | 90% | 99% |
| Aspergillius flavus | 60,000 | 99,000 |
| Aspergillius glaucus | 44,000 | 88,000 |
| Aspergillius niger | 132,000 | 330,000 |
| Mucor racemosus A | 17,000 | 35,200 |
| Mucor racemosus B | 17,000 | 35,200 |
| Oospora lactis | 5,000 | 11,000 |
| Penicillium expansum | 13,000 | 22,000 |
| Penicillium roqueforti | 13,000 | 26,400 |
| Penicillium digitatum | 44,000 | 88,000 |
| Rhisopus nigricans | 111,000 | 220,000 |
| Protozoa | 90% | 99% |
| Chlorella Vulgaris | 13,000 | 22,000 |
| Nematode Eggs | 45,000 | 92,000 |
| Paramecium | 11,000 | 20,000 |
| Virus | 90% | 99% |
| Bacteriopfage - E. Coli | 2,600 | 6,600 |
| Infectious Hepatitis | 5,800 | 8,000 |
| Influenza | 3,400 | 6,600 |
| Poliovirus - Poliomyelitis | 3,150 | 6,600 |
| Tobacco mosaic | 240,000 | 440,000 |
| Yeast | 90% | 99% |
| Brewers yeast | 3,300 | 6,600 |
| Common yeast cake | 6,000 | 13,200 |
| Saccharomyces carevisiae | 6,000 | 13,200 |
| Saccharomyces ellipsoideus | 6,000 | 13,200 |
| Saccharomyces spores | 8,000 | 17,600 |

A common problem of present UV disinfection systems and techniques is that they are stationary and can provide inadequate or incomplete disinfection of surfaces due to blockage of the UV radiation. For example, for disinfection of a hospital room, a UV device is brought in and placed in the center of the room for disinfection. Any furniture or appliances that are in the room may block the surfaces behind them from receiving an adequate dose of UV radiation, thus leading to ineffective disinfection of those surfaces. Current UV disinfection systems and techniques may also be bulky, lending them unable to be easily maneuvered to disinfect between tight or difficult-to-reach spaces. Moreover, current UV disinfection systems and techniques may have limited disinfection zone capacity, meaning that they are limited to smaller disinfection spaces, such as a hospital patient room or a hotel room.

To provide more effective disinfection of complex and large spaces, example apparatuses and methods for providing distribution of UV radiation are provided herein. As will be described in greater detail below, an apparatus for providing distribution of UV radiation may include a light-source housing assembly configured to emit UV radiation attached to an arm via the attachment component. The light-source housing assembly may include a directionalization cover for directing the UV radiation towards a desired disinfection zone. Additionally, the arm and/or attachment component may be pivotable or moveable such to allow for easy maneuverability of the light-source housing assembly. This can allow the assembly to direct UV radiation onto hard-to-reach surfaces and disinfect complex spaces.

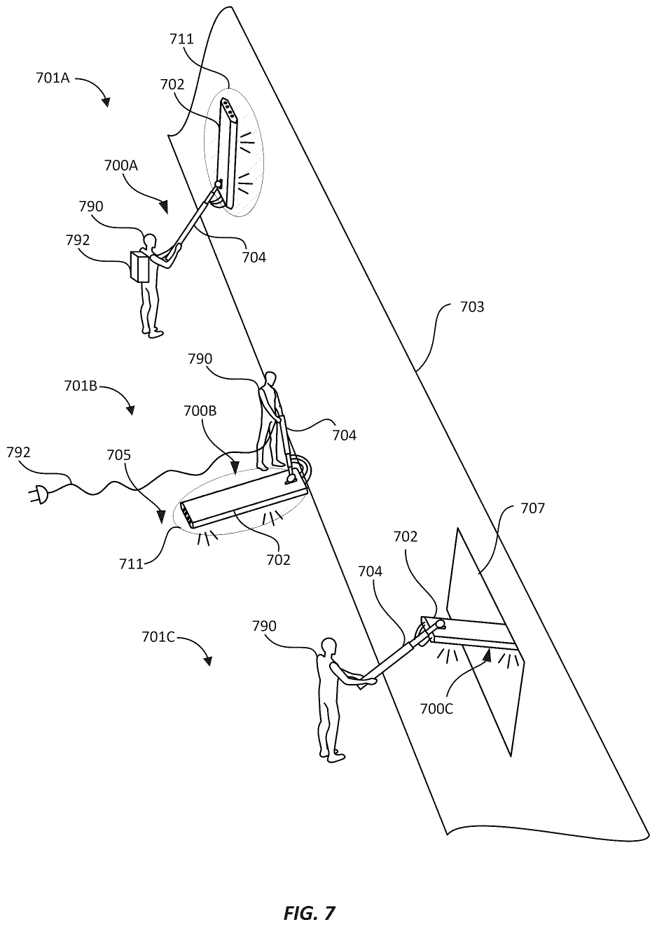

Additionally, as will be described in greater detail below with respect to FIGS. 7-10, the apparatus for distribution of UV radiation may be configurable to be handheld by a user or may be configurable to attached to a mobile device, such as a motorized vehicle. This can allow the apparatus to disinfect large areas or hard-to-reach areas, such as inside equipment or vessels, with minimal interruption to the disinfection process.

This illustrative example is given to introduce the reader to the general subject matter discussed herein and the disclosure is not limited to this example. The following sections describe various additional non-limiting examples and examples of systems, apparatuses, and methods for distributing UV radiation for disinfection purposes.

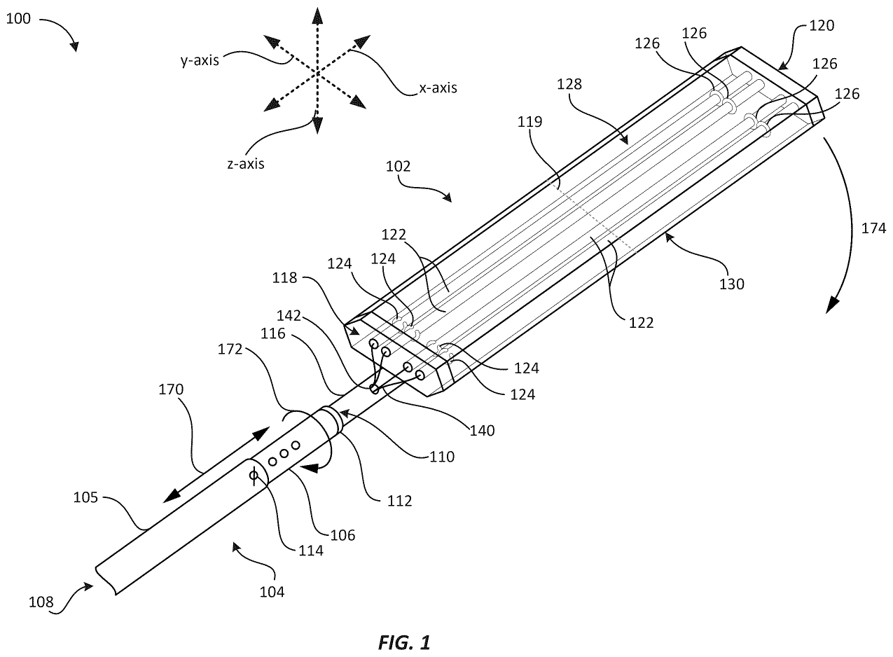

Referring now to FIG. 1, an example apparatus 100 for distributing UV radiation is provided, according to an embodiment herein. The apparatus 100 may include a light-source housing assembly 102 and an arm 104. The arm 104 may include a distal end 108 and a proximal end 110. The light-source housing assembly 102 may have a first end 118 and a second end 120. The proximal end 110 of the arm 104 may attach to the first end 118 of the light-source housing assembly 102. As will be described in greater detail below, in some embodiments, the proximal end 110 of the arm 104 may attach to the first end 118 of the light-source housing assembly 102 via an attachment component 116.

The light-source housing assembly 102 may also include one or more light sources 122 positioned within the light-source housing assembly 102 such to extend from the first end 118 to the second end 120 of the light-source housing assembly 102. Although the light sources 122 are illustrated as positioned to extend from the first end 118 of the light-source housing assembly 102 to the second end 120 of the light-source housing assembly 102, it should be appreciated that the light sources 122 may be positioned in the light-source housing assembly 102 having any other configuration, such as positioned to be perpendicular to the first end 118 and the second end 120 of the light-source housing assembly 102 or to be diagonal to the first end 118 and the second end 120 of the light-source housing assembly 102. The positioning of the light sources 122 within the light-source housing assembly 102 may depend on the configuration of the light-source housing assembly 102 (e.g., the light-source housing assembly 102 having a square shape as opposed to the depicted rectangular shape) or may depend on the size and shape of the light sources 122 themselves.

The light sources 122 may emit UV radiation when in a powered-on state. For example, the light sources 122 may include UV bulbs or lamps. In some embodiments, the light sources 122 may include a single UV bulb or lamp, while in other embodiments, the light sources 122 may include two or more UV bulbs or lamps, such as for example four UV bulbs as depicted in FIG. 1. As would be appreciated by those skilled in the art, the light sources 122 may include any number of UV bulbs or lamps, depending on the disinfection process and/or the configuration of the light-source housing assembly 102.

The light sources 122 may be sources capable of emitting ultraviolet-C (UVC) wavelengths or radiation. As understood by those skilled in the art, UVC radiation includes wavelengths of the light spectrum that are capable of inactivating viruses and bacteria, thereby disinfecting against pathogens. For example, the light sources 122 may emit wavelengths between 100-280 nm or between 200-280 nm. In some embodiments, the emittance wavelength of the light sources 122 may depend on a target pathogen of the disinfection process. As noted above, the size, shape, and pathology of a microorganism can impact the specific UV wavelength and duration of radiation required to inactivate the microorganism.

To power the light sources 122, the light-source housing assembly 102 may receive power from one or more wiring circuits 140 connected to a power source (not shown). The wiring circuits 140 may be connected to the power source such that when the power source supplies power, the light sources 122 emit UV radiation. The power source may include any known source of power such as a generator, photovoltaic cell, thermopiles, primary-cell batteries (e.g., battery pack), electrical outlet, electric motor, and the like. As will be described in greater detail below with respect to FIGS. 7-10, the power source may depend on the configuration, application, and/or portability of the apparatus 100.

To hold the light sources 122 in place, the light-source housing assembly 102 may include one or more light-source holder 124 and light-source holder 126. As illustrated, each of the light sources 122 may include a first light-source holder 124 at a first end of each of the light sources 122 and a second light-source holder 126 at a second end of each of the light sources 122. For example, if the light sources 122, as depicted, includes four UV bulbs, each of the UV bulbs may include a first light-source holder 124 at the first end of a UV bulb and a second light-source holder 126 at the second end of the UV bulb. Each of the four UV bulbs may include a first light-source holder 124 at the first end and a second light-source holder 126 at the second end. In another embodiment, a first light-source holder 124 may be configured to hold all four UV bulbs at the first end and the second light-source holder 126 may be configured to hold all four UV bulbs at the second end. In other words, in some embodiments, the first light-source holder 124 may be configured to hold a single light source 122 (e.g., UV bulb) at the first end and the second light-source holder 126 may be configured to hold the single light source 122 at the second end. While in other embodiments, the first light-source holder 124 may be configured to hold more than one light source 122 at the first end and the second light-source holder 126 may be configured to hold more than one light source 122 at the second end.

The light-source holder 124 and the light-source holder 126 may be or include a clasp, clip, or strap that is configured to receive and hold a light source 122 in position. For example, the light-source holder 124 and light-source holder 126 may include a C-clip or retention ring. In other embodiments, the light-source holder 124 and the light-source holder 126 may be or include threads configured to receive and hold the light source 122 in position. For example, the light-source holder 124 and the light-source holder 126 may be a socket into which a light source 122 screws into to receive power. It should be appreciated that the light-source holder 124 may include a different mechanism for holding the light source 122 in position than the light-source holder 126. For example, the light-source holder 124 may be a socket while the light-source holder 126 may be a clip or clasp.

As illustrated, the light-source holder 124 is positioned proximate to the first end 118 of the light-source housing assembly 102 and the light-source holder 126 is positioned proximate to the second end 120 of the light-source housing assembly 102. The light-source holder 124 may be positioned proximate to the first end 118 such that the light-source holder 124 is closer to the first end 118 than a mid-point 119 of the light-source housing assembly 102. Similarly, the light-source holder 126 may be positioned proximate to the second end 120 such that the light-source holder 126 is closer to the second end 120 than the mid-point 119 of the light-source housing assembly 102. It should be appreciated, however, that in some embodiments, one or both of the light-source holder 124 and the light-source holder 126 may be positioned so as to be proximate to the mid-point 119 (e.g., the light-source holder 124 being closer to the mid-point 119 than the first end 118 or the light-source holder 126 positioned closer to the mid-point 119 than to the second end 120). Additionally, in some embodiments, there may be a light-source holder (either 124 or 126) positioned at the mid-point 119. In still further embodiments, the light-source holder 124 and the light-source holder 126 may be part of a single light-source holder that extends from the first end 118 to the second end 120 of the light-source housing assembly 102, such as a slot into which a light source 122 is received and held in position.

To direct the emission of UV light from the light sources 122, the light-source housing assembly 102 may include a directionalization cover 130. The directionalization cover 130 may include one or more reflective materials. For example, the directionalization cover 130 may include a reflective coating or surface containing a reflective material. Reflective materials may include nanocrystalline metal oxides, such as titanium dioxide (TiO₂), zinc oxide (ZnO), magnesium oxide (MgO), or aluminum oxide (Al₂O₃), aluminum-containing materials, reflectors, polymers (HDPE), standard metals and metal alloys, stainless steel, polished metals, or mirrors.

In addition to or in place of the reflective material, the directionalization cover 130 may have a shape such to direct the UV radiation from the light sources 122. For example, the directionalization cover 130 may be concave about the light sources 122 such to focus the UV radiation from the light sources 122 onto a disinfection zone. Example shapes of the directionalization cover 130 are described in greater detail with respect to FIGS. 3A-3C.

The light-source housing assembly 102 may also include a transmittance cover 128. The transmittance cover 128 may be positioned opposite to the directionalization cover 130 such to allow transmission of the UV radiation, including the reflected portions of UV radiation, from the light sources 122 towards a disinfection zone. The disinfection zone may be an area for which the UV radiation from the apparatus 100 is directed and intended to disinfect. As will be described in greater detail below, in some embodiments, the apparatus 100 may include a timer or software that indicates a duration and/or intensity of UV radiation required to achieve disinfection of the disinfection zone. Disinfection may be achieved when at least 50%, at least 60%, at least 70%, at least 80%, at least 90%, or at least 95% of microorganisms within the disinfection zone are deactivated (e.g., killed). In some embodiments, disinfection may be pathogen specific (having a pathogen target); in such cases, disinfection may be achieved when at least 50%, at least 60%, at least 70%, at least 80%, at least 90%, at least 95%, or at least 99% of a target pathogen within the disinfection zone are deactivated.

The transmittance cover 128 may include a clear or translucent material or a material having a high transmission rate. For example, the transmittance cover 128 may include quartz glass, silica glass, and UV permeable polymer.

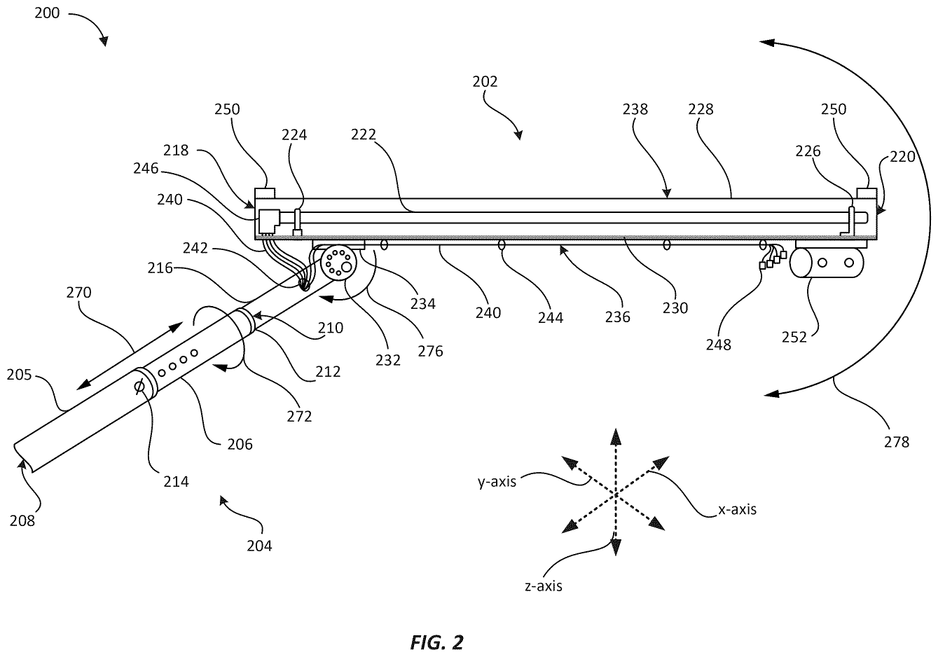

The light-source housing assembly 102 may be attached to the arm 104 at the first end 118. For example, the arm 104 may attach to the light-source housing assembly 102 via an attachment component 116. The attachment component 116 may be moveably coupled to a proximal end 110 of the arm 104 and moveably coupled to the first end 118 of the light-source housing assembly 102. In some embodiments, the attachment component 116 may only be moveably coupled to one of the proximal end 110 of the arm 104 or the first end 118 of the light-source housing assembly 102 while being fixed to the other. For example, the attachment component 116 may be moveably coupled to the proximal end 110 of the arm 104 while being fixed to the first end 118 of the light-source housing assembly 102. Various configurations of the attachment component 116 are described in detail with respect to FIGS. 2, 4, and 5.

As used herein, moveably coupled may mean that a respective component can rotate about one or more axes. In some embodiments, moveably coupled may mean that the respective component has or provides 6 degrees of freedom. For example, 6-degrees of freedom may mean that the respective component can move about three perpendicular axes, including changing orientation through rotation about the three perpendicular axes. The three perpendicular axes may include an x-axis, y-axis, and a z-axis, as commonly known in the art. An example set of perpendicular axes is illustrated on FIG. 1 for reference. Additionally, as used herein the term pivotable, such as a pivotable connection, may mean that the respective component provides a moveable coupling to any associated components. For example, a pivotable connection or component can provide 6 degrees of freedom to an attached or associated component.

The arm 104 may include a moveable element 114. For example, as illustrated, the arm 104 may include an extension component that allows the arm 104 to extend or move along the x-axis as indicated by movement arrow 170. In some embodiments, the moveable element 114 may be a pin-and-lock mechanism that allows a first segment 105 of the arm 104 to move with respect to a second segment 106 of the arm 104. As illustrated, the moveable element 114 may allow the second segment 106 to slide in and out of the first segment 105 to adjust the length of the arm 104. In other embodiments, the moveable element 114 may include one or more different extension components, such as a telescoping mechanism, hydraulic piston, motorize gear, and other robotic extending components.

In some embodiments, the moveable element 114 may allow the second segment 106 to be independently rotatable from the first segment 105. For example, the moveable element 114 may allow the second segment 106 to rotate about a first direction that is traverse to the x-axis, as indicated by movement arrow 272.

In some embodiments, the moveable element 114 may be or include a joint or a pivotable component 112. For example, in addition to or in place of an extension component, the moveable element 114 may include a pivotable component 112. The pivotable component 112 may allow the attachment component 116 to pivot or rotate about the x-axis, as indicated by the movement arrow 172, with respect to the second segment 106 or the arm 104. The x-axis may be considered to be a longitudinal axis of the arm 104. The pivotable component 112 may allow the light-source housing assembly 102 to rotate about the x-axis with respect to the arm 104, thereby allowing the apparatus 100 to reach and disinfect hard-to-reach areas.

In some embodiments, instead of or in addition to allowing for rotation of the light-source housing assembly 102 with respect to the arm 104, the pivoting component 112 may provide a pivoting or side-to-side movement along the y-axis with respect to the longitudinal axis of the arm 104. For example, the pivoting component 112 may allow the light-source housing assembly 102 to move along the y-axis, as indicated by the movement arrow 174, with respect to the arm 104. Again, by providing the light-source housing assembly 102 the ability to pivot and move with respect to the arm 104, the apparatus 100 may be able to access and disinfect hard to reach areas, such as on top of or under equipment. In other words, the pivoting component 112, moveable element 114, and/or attachment component 116 allow the apparatus 100 to adapt to the disinfection zone, thereby providing more complete and adequate disinfection of an area over conventional techniques.

In some embodiments, the attachment component 116 may include a port 142. The port 142 may be a hole through which one or more of the wiring circuits 140 extend. For example, the wiring circuits 140 may run through an interior of the arm 104 from the port 142 through the distal end 108 of the arm 104 to attach to a power source (not shown). By running the wiring circuits 140 through the interior of the arm 104, the wiring circuits 140 can be protected during a disinfection process (e.g., prevented from catching on equipment, objects in the area, or exposure to the environment).

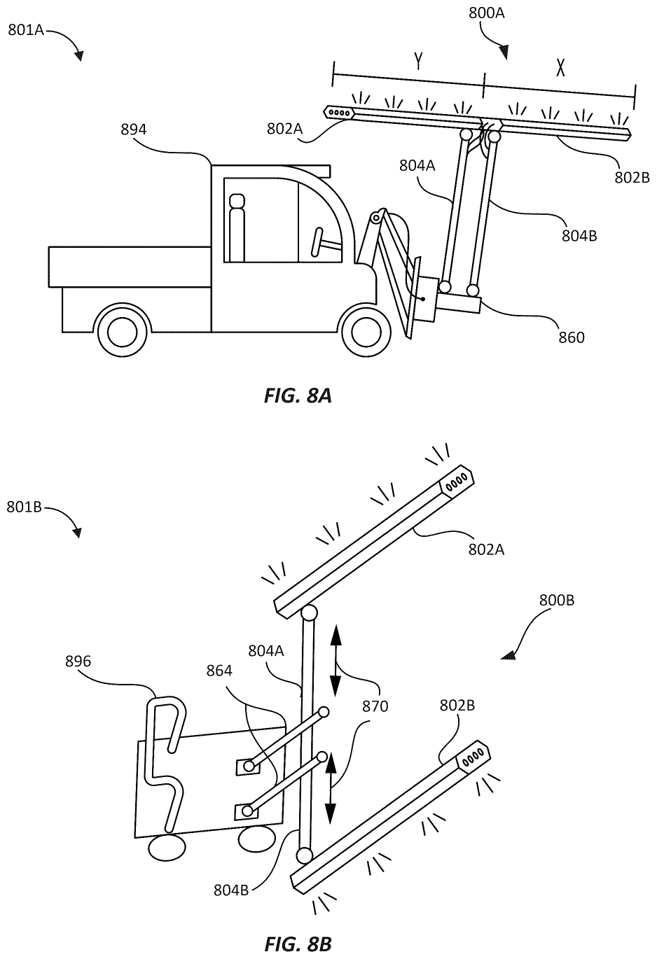

Although not shown, the distal end 108 of the arm 104 may attach to a handle. The handle may be configured to be held by a user and provide control of the apparatus 100 during a disinfection process. The handle may include a gripping component that is configured to provide friction and support of the apparatus by a user's hand. For example, the gripping component may conform to the user's hand and include anti-slip material. Additionally, as will be described below in greater detail with respect to FIGS. 8-10, the apparatus 100 may be attached to a motorized vehicle or mobile device.

Referring now to FIG. 2, another view of the example apparatus 200 for distributing UV radiation of FIG. 1 is provided, according to an embodiment herein. The apparatus 200 may be the same or similar to the apparatus 100. Similar numbering is used throughout the FIGURES to indicate the same or similar components. For example, a light-source housing assembly 202 may be the same or similar to the light-source housing assembly 102 having a first end 218 and a second end 220 that is the same or similar to the first end 118 and the second end 120, respectively, and an arm 204 may be the same or similar to the arm 104 having a distal end 208 and a proximal end 210 that is the same or similar to the distal end 108 and the proximal end 110, respectively.

Similar to the apparatus 100, the proximal end 210 of the arm 204 may attach to the light-source housing assembly 202 via an attachment component 216. The attachment component 216 may be the same or similar to the attachment component 116. As illustrated by FIG. 2, however, the attachment component 216 may attach the light-source housing assembly 202 to the arm 204 in a slightly different configuration than illustrated in FIG. 1. As depicted, the attachment component 216 may include a pivotable connection 232. The pivotable connection 232 may attach to the light-source housing assembly 202 proximate to the first end 218. In some embodiments, the pivotable connection 232 may attach to the light-source housing assembly 202 at the first end 218. The attachment of the pivotable connection 232 to the light-source housing assembly 202 may be considered to be attached at the first end 218 because the pivotable connection 232 attaches proximate to the first end 218.

The pivotable connection 232 may attach to a backside 236 of the light-source housing assembly 202. In some embodiments, the backside 236 may be opposite or opposing to the transmittance cover 228, which may be the same or similar to the transmittance cover 128. As described above, the backside 236 may include a directionalization cover 230, which may be the same or similar to the directionalization cover 130. The directionalization cover 230 may be on a surface of the backside 236 facing light sources 222. The light sources 222 may be the same or similar to the light sources 122. In some embodiments, the pivotable connection 232 may attach to the backside 236 of the light-source housing assembly 202 via a bolster 234. The bolster 234 may be a support or a component to provide additional security to the attachment of the pivotable connection 232 to the light-source housing assembly 202. As can be appreciated by those skilled in the art, as the light-source housing assembly 202 extends away from the attachment point of the pivotable connection 232, a strain may be created at the attachment point. The longer the configuration of the light-source housing assembly 202 (e.g., the greater the distance between the first end 218 and the second end 220 of the light-source housing assembly 202), the greater the strain may be at the attachment point. Accordingly, the bolster 234 may be configured to reduce or address the strain at the attachment point to avoid breakage or separation of the pivotable connection 232 from the light-source housing assembly 202 at the attachment point.

In some embodiments, the bolster 234 may also allow for rotation or pivoting of the light-source assembly 202 with respect to the arm 204. For example, the bolster 234 may allow rotation of the light-source assembly 202 along the z-axis. This can allow the light-source assembly 202 to pivot forward away from the arm 204.

The pivotable connection 232 may allow the light-source housing assembly 202 to be moveably coupled to the arm 104. For example, as depicted, the pivotable connection 232 may allow the light-source housing assembly 202 to pivot or move about the y-axis or an axis that is perpendicular to the x-axis, as indicated by the movement arrow 278. As noted above, the x-axis may be considered to be a longitudinal axis about which the arm 104 extends, as indicated by movement arrow 270. The pivotable connection 232 may rotate about a z-axis, as indicated by the movement arrow 276 to move the light-source housing assembly 202 along the movement arrow 278. The pivotable connection 232 may be or include a ratchet pivot, lockable pivot, or lockable hinge joint that allows for pivoting of the light-source housing assembly 202 and may also secure the light-source housing assembly 202 on a desired position. The pivotable connection 232 may be or include a mechanical, electrical, or hydraulically driven pivot.

Similar to apparatus 100, the light sources 222 may be secured within the light-source housing assembly 202 via a first light-source holder 224 proximate to the first end 218 and a second light-source holder 226 proximate to the second end 220. The light sources 222 may also be connected to a power source (not shown) via one or more wiring circuits 240. The wiring circuits 240 may be the same or similar to the wiring circuits 140 and may extend through an interior volume of the arm 204 via the port 242. As illustrated, positive ends of the wiring circuits 240 may connect into a socket 246 and include quick connects 248 for the wiring circuits 240 to allow connection with additional apparatus, specifically additional light-source housing assemblies (not shown). The wiring circuits 240 may be secured to the backside 236 of the light-source housing assembly 202 via attachments 244. The attachments 244 may be clips, clasps, straps, and the like.

The apparatus 200 may also include a connector 252. The connector 252 may allow attachment of another apparatus (not shown) to the light-source housing assembly 202. For example, in some embodiments, more than one apparatus 200 may be connected together to provide cover a larger disinfection zone, such as a floor or wall surface. In such cases, an arm of another apparatus that may be similar to the apparatus 200 may be attached to the light-source housing assembly 202 via the connector 252. In some embodiments, an additional light-source assembly may be connected directly to the light-source housing assembly 202 via the connection 252. Additional light-source housing assemblies, or in some cases additional apparatuses, may be joined consecutively to extend the disinfection zone of the apparatus 200. In some embodiments, up to 4 light-source housing assemblies or apparatus, up to 6 light-source housing assemblies or apparatus, or 10 light-source housing assemblies or apparatus may be connected, depending on the size and shape of the light-housing assemblies.

In some embodiments, the light-source housing assembly 202 may include one or more sensors 250. The sensors 250 may be positioned on a front side 238 of the light-source housing assembly 202. For example, the sensors 250 may be positioned on top of the transmittance cover 228. As depicted, a first sensor 250 may be positioned on the front side 238 proximate to the first end 218 and a second sensor 250 may be positioned on the front side 238 proximate to the second end 220. Although only two sensors 250 are depicted, it should be appreciated that any number of sensors 250 may be present. Additionally, in some embodiments, one or more sensors 250 may be positioned on the first end 218 and/or the second end 220. While the one or more sensors 250 are illustrated on the first end 218 and/or the second end 220, it should be appreciated that the one or more sensors 250 may be positioned on any surface of the light-source housing assembly 202, depending on the disinfection process and sensing requirements.

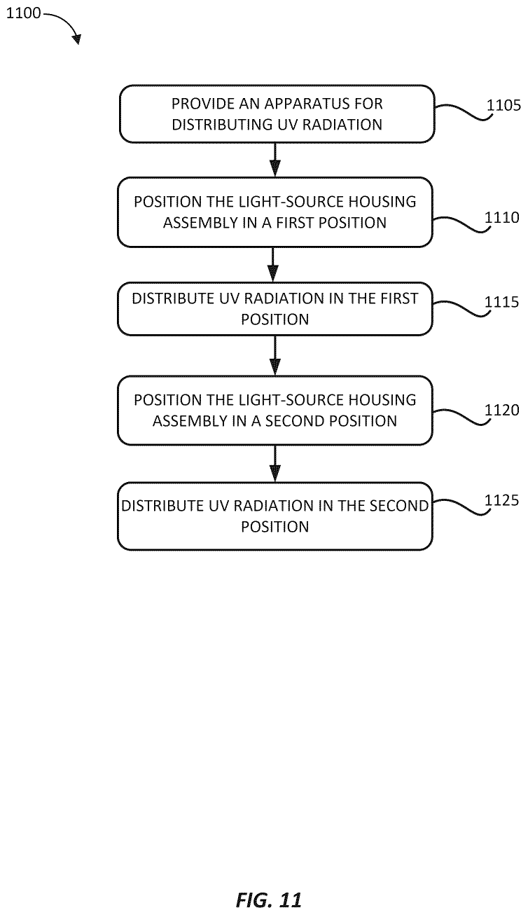

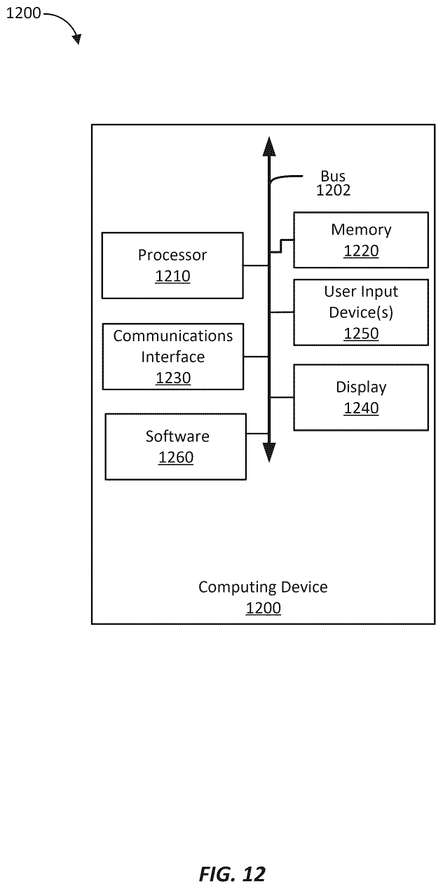

The sensors 250 may be or include a position sensor, a proximity sensor, or a camera. Since the apparatus 200 is used to disinfect hard-to-reach areas, a user of the apparatus 200 may often not be able to see the disinfection zone during the disinfection process. By having the sensors 250, the user can be provided with feedback as to a position of the light-source housing assembly 202 with respect to a disinfection zone. For example, the sensors 250 may provide positional feedback on a position of the light-source housing assembly 202 with respect to a disinfection zone or the sensors 250 may provide an image or video feed of the disinfection zone as viewed from the light-source housing assembly 202. As will be described in greater detail with respect to FIGS. 11 and 12, the sensors 250 may be used during an automatic disinfection process, providing feedback that the light-source housing assembly 202 is in a correct position with respect to the disinfection zone to achieve adequate disinfection of an area. Additionally, the sensors 250 may be used to indicate that the light-source housing assembly 202 is in the correct position for an adequate amount of time to achieve disinfection.

In addition to pivoting along the movement arrow 278, the apparatus 200 may also be moveable in one or more additional directions. For example, similar to the arm 104, the arm 204 may include a pivotable component 212 (the same or similar to the pivotable component 112) that allows for rotation of the attachment component 216 with respect to the arm 204 about the x-axis, as indicated by movement arrow 272. Additionally, the arm 204 may include a moveable element 214, which is the same or similar to the moveable element 114, that allows for movement of the arm along the x-axis, as indicated by the movement line 270.

Referring now to FIGS. 3A-3C, example cross-sectional views of a light-source housing assembly are provided, according to an embodiment herein. As noted above, the same or similar numbering may be used to reference the same or similar components across the FIGURES. For example, the light-source housing assemblies 302A, 302B, or 302C may be the same or similar to the light-source housing assembly 102 or 202. Each of the light-source housing assemblies 302A, 302B, and 302C depict a cross-sectional view of a different configuration of a light-source housing assembly. For example, the cross-sectional views provided in FIGS. 3A, 3B, and 3C may be taken at a mid-point of the light-source housing assembly, such as for example, at the mid-point 119. As noted above, the light-source housing assembly may have a different configuration such to directionalize UV radiation from the light sources, such as light sources 322.

Starting with FIG. 3A, the light-source housing assembly 302A may include a front side 338 and a backside 336. The front side 338 may include a transmittance cover 328 that is similar or the same as the transmittance cover 128 or 228. The backside 336 may include a directionalization cover that may be the same or similar to the directionalization cover 130 or 230. As illustrated, the directionalization cover may be formed by three directionalization surfaces: a back surface 330a, a first side surface 330b, and a second side surface 330c. The back surface 330a may be parallel to the backside 336 of the light-source housing assembly 302A. The first side surface 330a and the second side surface 330c may be perpendicular to the back surface 330a. Due to the varying orientations of the surfaces 330a, 330b, and 330c, the back surface 330a may direct emittance of UV radiation from the light sources 322 in a first direction, the first side surface 330b may direct emittance of UV radiation in a second direction, and the second surface 330c may direct emittance of UV radiation in a third direction. In some embodiments, the first direction, the second direction, and the third direction may be different directions from each other.

In some embodiments, the first side surface 330b and the second surface 330c may be at a non-perpendicular angle to the back surface 330a. For example, turning to FIG. 3B, the light-source housing assembly 302B depicts a configuration in which the first side surface 330b and the second side surface 330c are oriented at a non-perpendicular angle to the back surface 330a. As should be appreciated by those skilled in the art, varying the orientation of the first side surface 330b and the second side surface 330c with respect to the back surface 330a may impact the reflection path of UV radiation when emitted from the light sources 322.

Turning now to FIG. 3C, in some embodiments the backside 336 of the light-source housing assembly may be curved, such as illustrated by the light-source housing assembly 302C. In such cases, the directionalization cover 330 may be curved as well. For example, the directionalization cover 330 may be concave about the light sources 322 such to direct UV radiation from the light sources 322 towards a focus area through the transmittance cover 328.

Although the light-source housing assemblies 302A, 302B, and 302C illustrate various configurations for a light-source housing assembly and directionalization covers 330, it should be appreciated that any variation or configuration is contemplated herein.

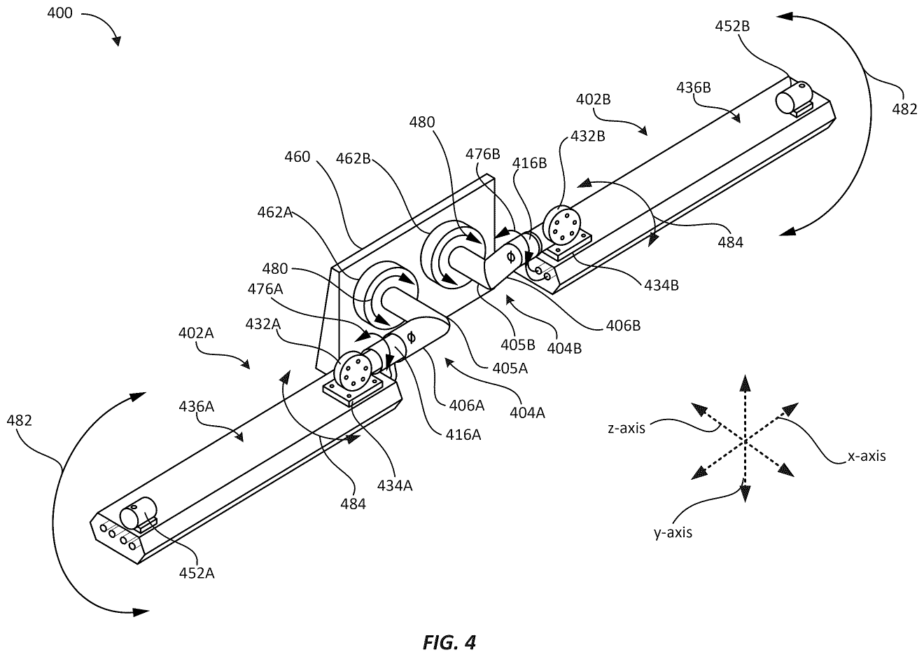

In some embodiments, an apparatus for distribution UV radiation, such as the apparatus or 200, may include more than one light-source housing assembly. Referring now to FIG. 4, an example apparatus 400 for distributing UV radiation having two light-source housing assemblies 402A and 402B is illustrated, according to an embodiment herein. The light-source housing assemblies 402A and 402B may be the same or similar to the light-source housing assembly 102, 202, or any of 302A, 302B, and 302C, including any of the components therein.

As illustrated by FIG. 4, apparatus 400 may include a first light-source housing assembly 402A and a second light-source housing assembly 402B. One or both of the first light-source housing assembly 402A and the second light-source housing assembly 402B may be operably connected to a bolster 460. The bolster 460 may be a plate, beam, or other component to which one or more of the first light-source housing assembly 402A and the second light-source housing assembly 402B are connected. In some embodiments, the bolster 460 may be configured to connect to a mobile device, such as a motorized vehicle. Various examples of mobile device to which the bolster 460 may be connected are discussed in greater detail with respect to FIGS. 8-10. In other embodiments, the bolster 460 may be configured to connect to a handling arm (not shown) for manual handling of the apparatus 400.

The first light-source housing assembly 402A may be operably connected to the bolster 460 via a first arm 404A. The first arm 404A may include a first segment 405A and a second segment 406A. The first segment 405A may be coupled to the bolster 460 via a movement element 462A. Similarly, the second light-source housing assembly 402B may be operably connected to the bolster 460 via a second arm 404B. The second arm 404B may include a first segment 405B and a second segment 406B. The first segment 405B may be coupled to the bolster 460 via a movement element 462B. As illustrated, each of the movement elements 462A and 462B may provide for movement of the first arm 404A and the second arm 404B, respectively. For example, the movement elements 462A and 462B may rotate along the movement arrow 480 to allow for movement of the first arm 404A and the second arm 404B along the y-axis, as indicated by the movement arrows 482. In other embodiments, the movement elements 462A and 462B may also provide movement or rotation of the first arm 404A and the second arm 404B along the x-axis or z-axis.

The first light-source housing assembly 402A may be moveably coupled to the second end 406A of the first arm 404A. For example, the first light-source housing assembly 402A may be moveably coupled to a proximal end of the first arm 404A via an attachment component 416A. Similar to attachment component 116, the attachment component 416A may allow for movement of the first light-source housing assembly 402A with respect to the first arm 404A, by for example, rotation or extension along the x-axis. In some embodiments, the attachment component 416A may be operably coupled to a pivotable connection 432A. The pivotable connection 432A, similar to the pivotable connection 332, may allow for pivoting or rotation of the first light-source housing assembly 402A with respect to the first arm 404A. For example, the pivotable connection 432A may allow for rotation of the light-source housing assembly 402A with respect to the first arm 404A about the x-axis as illustrated by movement arrow 476A.

Similarly, the second light-source housing assembly 402B may be moveably coupled to the second end 406B of the second arm 404B. For example, the second light-source housing assembly 402B may be moveably coupled to a proximal end of the second arm 404B via an attachment component 416B. The attachment component 416B may allow for movement of the second light-source housing assembly 402B with respect to the second arm 404B by rotation or extension along the x-axis. In some embodiments, the attachment component 416B may be operably coupled to a pivotable connection 432B, which allows for pivoting or rotation of the second light-source housing assembly 402B with respect to the second arm 404B about the x-axis, as illustrated by the movement arrow 476B. In some embodiments, the pivotable connection 432A may allow for rotation or movement of the first light-source housing assembly 402A along the z-axis or along the y-axis. Similarly, the pivotable connection 432B may allow for rotation or movement of the second light-source housing assembly 402B along the z-axis or along the y-axis. In an example embodiment, the pivotable connection 432 and the pivotable connection 432B may provide 6-degrees of freedom for the movement of the first light-source housing assembly 402A and the second light-source housing assembly 402B, respectively.

Depending on the orientation of the first light-source housing assembly 402A and the second light-source housing assembly 402B, the pivotable connection 432A may be coupled to a backside 436A of the first light-source housing assembly 402A via a plate 434A. Similarly, the pivotable connection 432B may be coupled to a backside 436B of the second light-source housing assembly 402B via a plate 434B. In some embodiments, the plates 434A and 434B may be rotatably connected to the backsides 436A and 436B, respectively, such to allow rotation of the first light-source housing assembly 402A and the second light-source housing assembly 402B about the y-axis, as illustrated by movement arrows 484.

In some embodiments, one or both of the first light-source housing assembly 402A and the second light-source housing assembly 402B may include a first connectors 452A and a second connector 452B, respectively. The first and second connectors 452A and 452B may be the same or similar to the connector 252. For example, the first and second connectors 452A and 452B may allow for additional apparatuses or light-source assemblies to be attached to the first light-source housing assembly 402A and the second light-source housing assembly 402B, respectively.

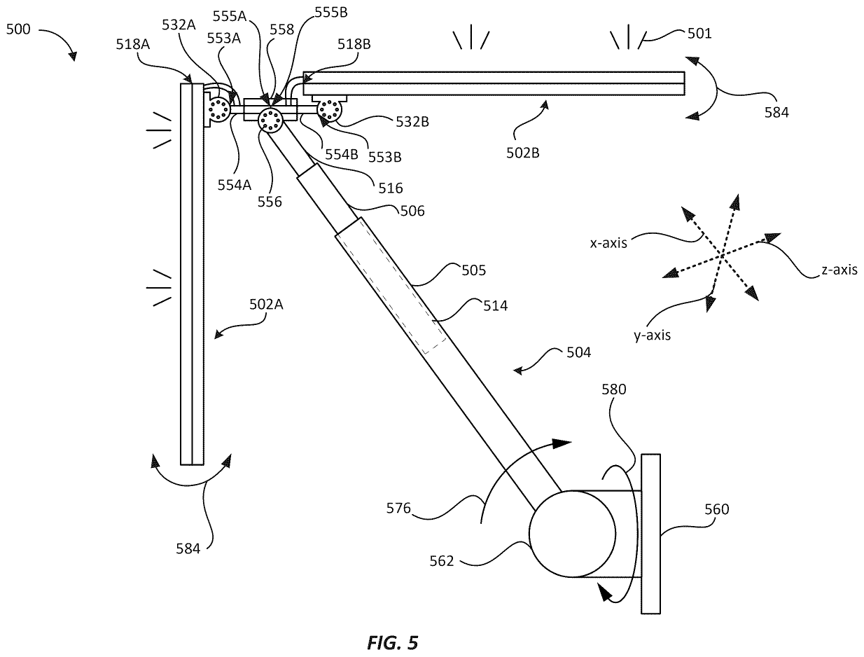

Referring now to FIG. 5, another example apparatus 500 for distributing UV radiation is illustrated, according to an embodiment herein. Apparatus 500 provides another example arrangement including two light-source housing assemblies 502A and 502B. The light-source housing assemblies 502A and 502B may be the same or similar to the light-source housing assembly 102, 202, any of 302A, 302B, 302C, 402A, or 402B including any of the components therein. Similar label numbering is used to indicate similar or the same components.

As illustrated, the apparatus 500 may include a first light-source housing assembly 502A and a second light-source housing assembly 502B. The first light-source housing assembly 502A and the second light-source housing assembly 502B may be operably connected to an arm via one or more stub arms 554A and 554B. In some embodiments, the first stub arm 554A and the second stub arms 554B may be separate components, however, in some embodiments the first stub arm 554A and the second stub arm 554B may be a single component.

The first stub arm 554A may have a first end 553A and a second end 555A. Similarly, the second stub arm 554B may include a first end 553B and a second end 555B. The first end 553A of the first stub arm 554A may be operably coupled to the first end 518A of the first light-source housing assembly 502A. In some embodiments, a pivotable connection 532A may couple the first end 553A of the first stub arm 554A to the first end 518A of the first light-source housing assembly 502A. As illustrated, coupled to the first end 518A may mean that the first stub arm 554A is coupled proximate to the first end 518A.

Similarly, the second stub arm 554B may be operably coupled to a first end 518B of the second light-source housing assembly 502B. In some embodiments, a pivotable connection 532B may couple the first end 553B of the second stub arm 554B to the first end 518B of the second light-source housing assembly 502B. The pivotable connection 532A and the pivotable connection 532B may allow the first light-source housing assembly 502A and the second light-source housing assembly 502B, respectively, to rotate about a y-axis, as indicated by the movement arrows 584. This can allow the first light-source housing assembly 502A and the second light-source housing assembly 502B to rotate and pivot as needed during a disinfection process. In other cases, the pivotable connections 532A and 532B may allow the first light-source housing assembly 502A and the second light-source housing assembly 502B to fold into the arm 504, thereby allowing the apparatus 500 to collapse into a compact form for ease of transportation and storage.

In some embodiments, a component 558 may be provided over the second ends 555A and 555B of the first stub arm 554A and the second stub arm 554B, respectively. The component 558 may be a cover or a supportive piece that supports the connection of the first stub arm 554A and the second stub arm 554B with the arm 504.

The arm 504 may include a first segment 505 and a second segment 506. As illustrated, the arm 504 may also include a moveable element 514 that allows for extension of the arm 504. For example, the moveable element 514 may allow for the second segment 506 and the first segment 505 to form a telescoping arm. The arm 504 may also include an attachment component 516. The attachment component 516 may be operably coupled to the first stub arm 554A and the second stub arm 554B via a pivotable connection 556. The pivotable connection 556 may allow the first stub arm 554A and the second stub arm 554B to be independently rotatable with respect to the arm 504. For example, the pivotable connection 556 may allow for at least 180° rotation of first stub arm 554A or the second stub arm 554B with respect to the arm 504.

The arm 504 may be operably connected to a bolster 560. For example, a pivotable connection 562 may connect the distal end of the arm 504 to the bolster 560. The pivotable connection 562 may allow for the arm 504 to be rotatably independent from the bolster 560. For example, the pivotable connection 562 may allow for the arm 504 to rotate about the x-axis, as indicated by the movement arrow 576, and the pivotable connection 562 may allow for the arm to rotate along one or both of the y-axis and the z-axis, as indicated by the movement arrow 580. In other words, the pivotable connection 562 may provide 6-degrees of freedom for movement of the arm 504

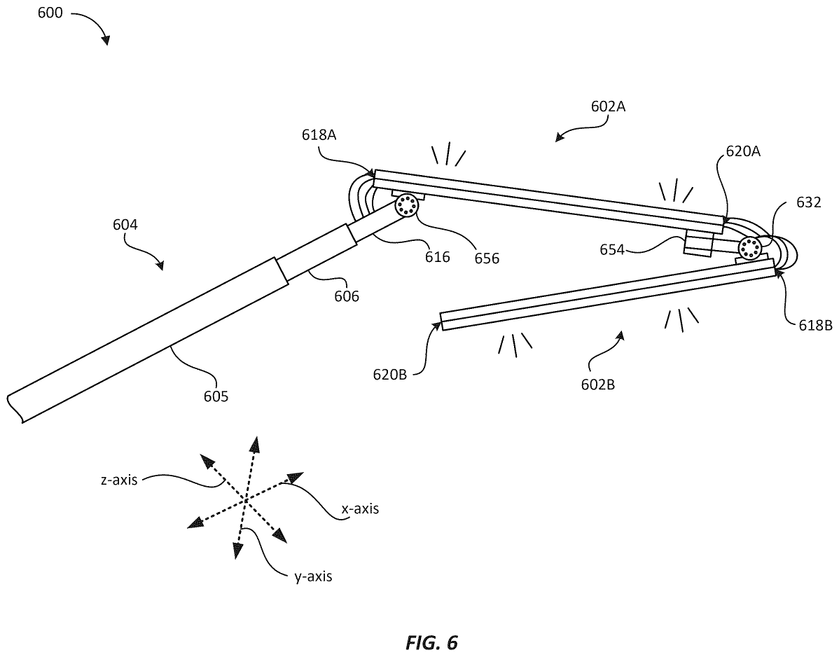

In some embodiments, the first light-source housing assembly and the second light-source housing assembly may be operably coupled to each other. Referring now to FIG. 6, another example apparatus 600 for distributing UV radiation having two light-source housing assemblies 602A and 602B is illustrated, according to an embodiment herein. The light-source housing assemblies 602A and 602B may be the same or similar to the light-source housing assembly 102, 202, any of 302A, 302B, 302C, 402A, 402B, 502A, or 502B including any of the components therein. Similar label numbering is used to indicate similar or the same components.

As illustrated, the apparatus 600 may include the first light-source housing assembly 602A and the second light-source housing assembly 602B. The first end 618A of the first light-source housing assembly 602A may be coupled to the arm 604. For example, the first end 618A may be operably coupled to a pivotable connection 656 of the arm 604. The pivotable connection 656 may be part of or coupled to an attachment component 616 of the arm 604. As illustrated, the arm 604 may include a first segment 605 and a second segment 606. As described above, the pivotable connection 656 may allow the first light-source housing assembly 602A to rotate about a y-axis or an axis that is transverse to a longitudinal axis of the arm 604.

The second light-source housing assembly 602B may be operably connected to the first light-source housing assembly 602A. As illustrated, a first end 618B of the second light-source housing assembly 602B may be coupled to a second end 620A of the first light-source housing assembly 602A. To couple the first light-source housing assembly 602A to the second light-source housing assembly 602B, a stub arm 654 may be provided. The stub arm 654 may couple the second end 620A of the first light-source housing assembly 602A to the first end 618B of the second light-source housing assembly 602B. In some embodiments, a pivotable connection 632 may couple to either the second end 620A of the first light-source housing assembly 602A or the first end 618B of the second light-source housing assembly 602B. The pivotable connection 632 may allow for the second light-source housing assembly 602B to be rotatably independent from the first light-source housing assembly 602A. As illustrated, the pivotable connection 632 may allow the second light-source housing assembly 602B to rotate about a y-axis. The rotatability of the second light-source housing assembly 602B with respect to the first light-source housing assembly 602A, and the rotatability of the first light-source housing assembly 602A with respect to the arm 604 allows for the apparatus 600 to be configurable to a disinfection zone. For example, the first light-source housing assembly 602A and the second light-source housing assembly 602B can be reoriented and positioned to distribute UV radiation onto hard-to-reach surfaces, conforming to the disinfection space or area. Additionally, the second light-source housing assembly 602B can collapse or fold into the first light-source housing assembly 602A, and the first light-source housing assembly 602A can collapse or fold into the arm 604, thereby allowing for the apparatus 600 to be compact for transportation and storage purposes.

As noted above, the apparatuses discussed herein, such as apparatuses 100, 200, 400, 500, and 600, may be used in a variety of environments for disinfection purposes. Example environments that the apparatuses provided herein may be employed in for disinfection purposes include hospital environments, such as patient rooms or operating rooms, chemical and manufacturing facilities, such as processing plants, agricultural facilities, such as poultry buildings, educational facilities, gyms, stadiums, airplanes or other transportation vehicles, sewers or water-treatment facilities, and the like. As could be appreciated, the apparatuses provided herein can be used in virtually any environment in which microorganisms exist.

The moveability, rotatability, and flexibility of the apparatus provided herein can allow the apparatus to conform to a disinfection area. As discussed above, the light-source housing assembly can be rotated to orient in a variety of directions, such to direct UV radiation emittance onto a desired disinfection zone. Additionally, the arm can extend, pivot, or otherwise move to direct the UV radiation onto hard-to-reach surfaces. Turning now to FIGS. 7-10, a variety of user embodiments and scenarios are provided to illustrate the numerous configurations and applications for which the apparatuses provided herein can be employed. The use-embodiments provided in the following FIGURES are not meant to be limiting and instead to provide illustrative examples for descriptive purposes. Those skilled in the art will readily appreciate the various configurations and embodiments for which the apparatuses herein can be used.

Referring now to FIG. 7, example use-configurations 701A, 701B, and 701C for disinfecting surfaces using an example apparatus for distributing UV radiation are illustrated, according to an embodiment herein. A first use-configuration 701A is provided. As illustrated, a user 790 can use an apparatus 700A for distribution of UV radiation. The apparatus 700A may be the same or similar to any of the apparatuses 100, 200, 400, 500, or 600. For example, the apparatus 700A may include an arm 704 and a light-source housing assembly 702, which may be the same or similar to any of the arms and light-source assemblies described herein.

In the first use-configuration 701A, the apparatus 700A may be used to disinfect a disinfection zone 711 on a wall surface 703. To disinfect the wall surface 703, the user 790 may extend the light-source housing assembly 702 using the arm 704. The user 790 can also orient the light-source housing assembly 702 such to direct the UV radiation onto the wall surface 703. The user 790 may be able to walk or otherwise move the light-source housing assembly 702 across the wall surface 703, thereby disinfecting a large disinfection zone.

The apparatus 700A may be powered via a portable battery pack 792. As illustrated, the portable battery pack 792 may be wearable by the user 790 such to allow for mobility and flexibility of the apparatus 700A during the disinfection process.

A second use-configuration 701B is provided. As illustrated, in the second use-configuration 701B the user 790 may use an apparatus 700B to disinfect the disinfection zone 711 on a floor surface 705. The apparatus 700B may be the same or similar to the apparatus 700A. The user 790 may reorient the light-source housing assembly 702 such to direct the UV radiation onto the floor surface 705. The arm of the apparatus 700B may be adjusted such to allow the user 790 to manipulate and move the light-source housing assembly 702 to disinfect a large area of the floor surface 705 with minimal discomfort.

In the use-configuration 701B, the apparatus 700B may be powered via an outlet source. For example, the apparatus 700B may include an outlet cord 792 for supplying power to the apparatus 700B. The outlet cord 792 may be configured to plug into an outlet source to supply power to the apparatus 700B.

A third use-configuration 701C is provided. As illustrated, in the third use-configuration 701C, the user 790 may use an apparatus 700C to disinfect inside a space 707. The apparatus 700C may be the same or similar to the apparatus 700A or the apparatus 700B. The space 707 may be a hole in the wall surface 703, may be a cubby or shelf, may be the inside of a piece of machinery, or any other hard-to-reach surface. To disinfect inside the space 707, the user 790 may reorient the light-source housing assembly 702 such to extend inside the space 707. The arm 704 may be adjusted to allow the user 790 to position the light-source housing assembly 702 inside the space 707.

In the use-configuration 701C, the apparatus 700C may include a rechargeable battery pack. For example, the apparatus 700C may be chargeable such to hold a charge during a disinfection process.

As discussed above, in some embodiments an example apparatus provided herein may be configurable to attach to a mobile device. Referring now to FIGS. 8A and 8B, example use-configurations 801A and 801B for using apparatuses for distributing UV radiation in combination with a motorized vehicle are illustrated, according to an embodiment herein. As illustrated by use-configuration 801A of FIG. 8A, an apparatus 800A may be coupled to a motorized vehicle 894. For example, the apparatus 800A may be attached to a front end of the motorized vehicle 894 via a bolster 860, which may be similar to the bolster 460 or 560. The apparatus 800A may include one or more arms 804A and 804B that allow for maneuvering of a first light-source housing assembly 802A and a second light-source housing assembly 802B. The first light-source housing assembly 802A may be independently moveable from the second light-source housing assembly 802B, such to allow the first light-source housing assembly 802A to emit UV radiation via zone Y and the second light-source housing assembly 802B to emit UV radiation via zone X. In some embodiments, the zone Y may be different than the zone X. In other words, the first light-source housing assembly 802A can be positioned in at a different orientation and arrangement than the second light-source housing assembly 802B such to emit UV radiation at a different angle than UV radiation emitted from the second light-source housing assembly 802B.

In the use-configuration 801A, the arms 804A and 804B, and the light-source housing assemblies 802A and 802B may be controllable by a driver of the motorized vehicle 894. For example, the motorized vehicle 894 may have a control panel (not shown) inside that allows a driver to control the positioning and orientation of the apparatus 800A. In some embodiments, the motorized vehicle 894 (e.g., the engine) may provide a power-source for the apparatus 800A.

A use-configuration 801B is provided by FIG. 8B, illustrating another example mobile device. As shown, an apparatus 800B may be coupled to a pushcart 896. The apparatus 800B may include two or more light-source housing assemblies 802A and 802B. The apparatus 800B may couple to the pushcart 896 via one or more posts 864. A first arm 804A and a second arm 804B may be coupled to the posts 864. As illustrated, the first and second arms 804A and 804B may be extendible, as indicated by movement arrow 870, such to allow the light-source housing assemblies 802A and 802B to emit UV radiation onto hard-to-reach surfaces.

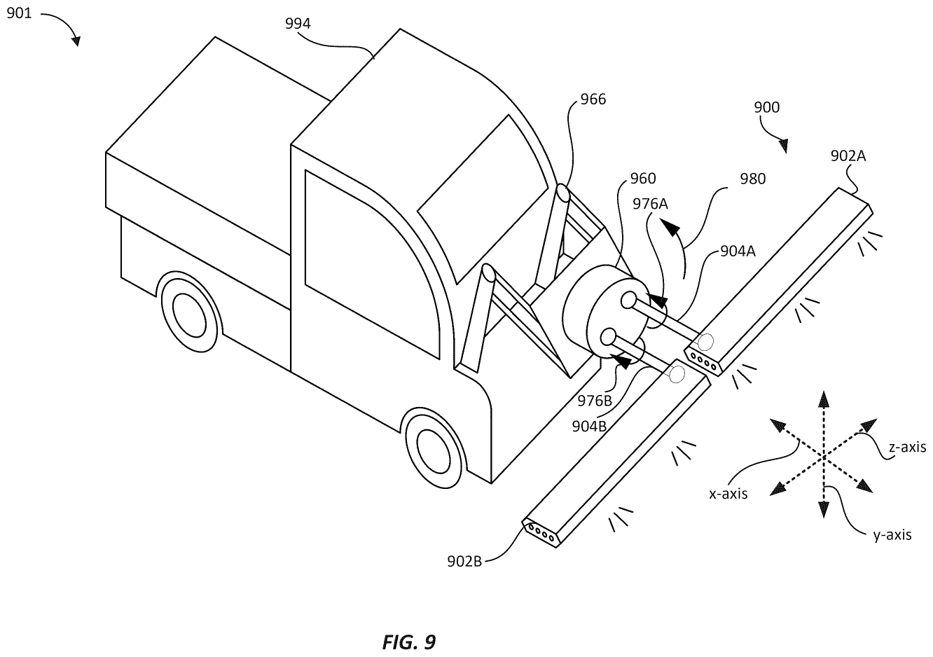

Referring now to FIG. 9, another example use-configuration 901 for using an apparatus 900 for distributing UV radiation in combination with a motorized vehicle 994 is illustrated, according to an embodiment herein. As illustrated by the use-configuration 901, the apparatus 900 may be coupled to the front of the motorized vehicle 994. The apparatus 900 may be coupled to the motorized vehicle 994 via a bolster 960. The bolster 960 may be rotatable or pivotable such to rotate about an x-axis, as indicated by movement arrow 980. Rotating the bolster 960 may rotate both of a first light-source housing assembly 902A and a second light-source housing assembly 902B of the apparatus 900. In some embodiments, a first arm 904A and a second arm 904B may be independently rotatable from each other. For example, the first arm 904A may be rotatable about the x-axis, as indicated by movement arrow 976A and the second arm 904B may be rotatable about the x-axis, as indicated by movement arrow 976B. This may allow the first arm 904A to rotate to orient the first light-source housing assembly 902A in a first direction or towards a first disinfection zone while the second arm 904B rotates to orient the second light-source housing assembly 902A in a second, different direction or towards a second disinfection zone. The motorized vehicle 994 may also include a lift 966 to which the bolster 960 is coupled such to allow the apparatus 900 to be lifted and directed towards various disinfection zones.

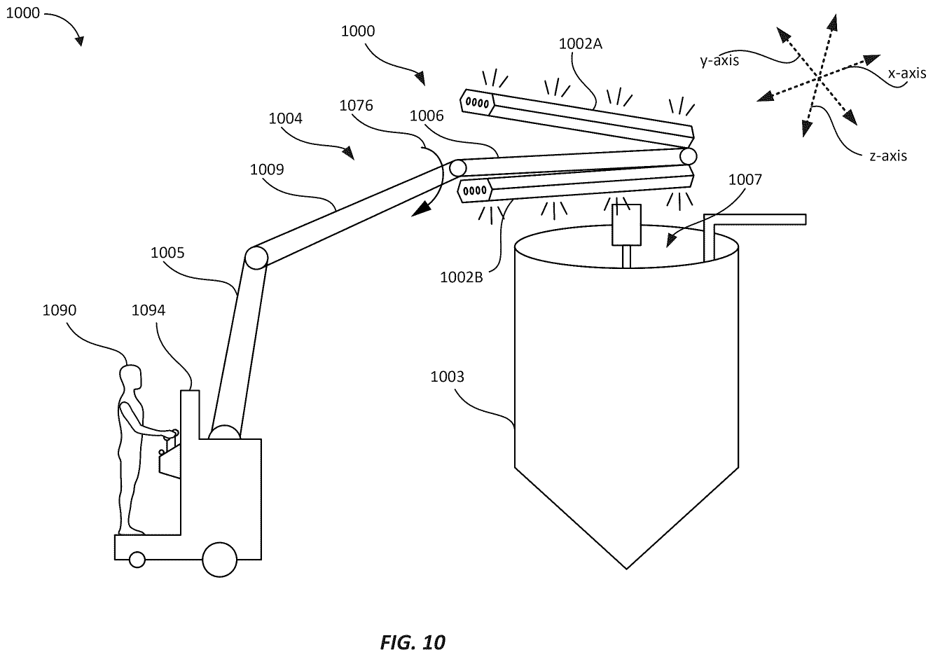

Referring now to FIG. 10, another example use-configuration 1001 using an example apparatus 1000 for distributing UV radiation in combination with a motorized vehicle 1094 is illustrated, according to an embodiment herein. As illustrated, the apparatus 1000 may be used to disinfect equipment 1003. The equipment 1003 may be a tank or mixer that is part of a manufacturing process. The motorized vehicle 1094 may be a lift that is configured to lift the apparatus 1000 to reach an inside space 1007 of the equipment 1003. As illustrated, the arm 1004 may include three segments, a first segment 1005, a second segment 1006, and a third, middle segment 1009. By increasing the number of segments of the arm 1004, the apparatus 1000 can extend its reach. It should be appreciated that any number of segments could be included in the apparatus 1000, however, for illustrative purposes only three segments are depicted.

As illustrated, the three segments 1005, 1006, and 1009 allow for light-source housing assemblies 1002A and 1002B to be cantilevered over the equipment 1003, thereby allowing distribution of UV radiation onto the inside space 1007. In some embodiments, the arm 1004 may be pivotable or rotatable about the x-axis, as indicated by movement arrow 1076, such to allow one or more of the light-source housing assembly 1002A and 1002B to be inserted into the inside space 1007 of the equipment 1003 for disinfection. As discussed in the examples provided above, the arm 1004 may be pivotable or rotatable about any axis, depending on the configuration of the apparatus 1000.

Referring now to FIG. 11, a flowchart of an example method 1100 for distributing UV radiation is provided, according to an embodiment herein. Specifically, the method 1100 may be for disinfecting a surface using UV radiation. The description of the method 1100 in FIG. 11 will be made with reference to FIGS. 1-10, however any suitable system according to this disclosure may be used.

The method 1100 may include step 1105. At step 1105, a light-source housing assembly may be provided. For example, a light-source housing assembly such as the light-source housing assembly 100, 200, 400, 500, 600, 700A, 700B, 700C, 800A, 800B, 900, or 1000 may be provided. As described above, the light-source housing assembly may have a first end and a second end and include at least one UV bulb operable to emit UV radiation, a first light-source holder configured to hold a first end of the at least one UV bulb within the light-source housing assembly, wherein the first light-source holder is positioned proximate to the first end of the light-source housing assembly. The light-source housing assembly may also include a second light-source holder configured to hold a second end of the at least one UV bulb within the light-source housing assembly, wherein the second light-source holder is positioned proximate to the second end of the light-source housing assembly. In some embodiments, the light-source housing assembly may include a directionalization cover extending from the first end of the light-source housing assembly to the second end of the light-source housing assembly, and a transmittance cover extending from the first end of the light-source housing assembly to the second end of the light-source housing assembly. The directionalization cover may include at least one surface for directing emittance of UV light from the at least one UV bulb and the transmittance cover may be configured to transmit the UV light from the at least one UV bulb onto a disinfection zone. The light-source housing assembly may also include a power source configured to provide power to the at least one UV bulb of the light-source housing assembly, an arm having a distal end and a proximal end, and including a moveable element, and an attachment component. The attachment component may be moveably coupled to the proximal end of the arm and moveably coupled to the first end of the light-source housing assembly.

The method 1100 may also include steps 1110 and 1115. At step 1110, the method 1100 may include positioning the light-source housing assembly in a first position relative to the arm. For example, step 1110 may include directionalizing the UV radiation from the at least one UV bulb towards a first disinfection zone. At step 1115, the method 1100 may include distributing UV radiation using the light-source housing assembly in the first position. For example, UV radiation may be distributed using the light-source housing assembly onto the first disinfection zone.

The method 1100 may also include steps 1120 and 1125. At step 1120, the method 1100 may include positioning the light-source housing assembly in a second position relative to the arm. For example, step 1120 may include directionalizing the UV radiation from the at least one UV bulb towards a second disinfection zone. The second disinfection zone may be different from the first disinfection zone. At step 1125, the method 1100 may include distributing UV radiation using the light-source housing assembly in the second position.

As described above, the amount of UV radiation distributed onto a disinfection zone may depend on the type of microorganism. The amount of UV radiation, which can include the duration of time that the light-source housing assembly is positioned over a disinfection zone, can vary depending on a distance that the light-source housing assembly is positioned from the disinfection zone. For example, when the light-source housing assembly is positioned near the disinfection zone, the amount of UV radiation distributed onto the disinfection zone may be greater than the amount of UV radiation distributed onto the disinfection zone when the light-source housing assembly is positioned further away from the disinfection zone.

Table 2 below provides example UV radiation dosages that are achievable at varying distances from a disinfection zone with an apparatus having four UV bulbs. As illustrated, when a light-source housing assembly containing the four UV bulbs was positioned at a distance of two inches from the disinfection zone, the apparatus can apply a 34,354 μW/cm² dosage of UV radiation onto the disinfection zone. As the distance between the light-source housing assembly and the disinfection zone is increased, the amount of UV radiation dosage decreases. At a distance of 12 inches, however, the apparatus still provides a dosage over 9000 μW/cm² of UV radiation. The UV radiation dosage is per second (time) of exposure.

TABLE 2

| Distance from | |
|---|---|
| Disinfection Zone | UV Radiation Dosage |
| (Inches) | (μW/cm²) |
| 2 | 34354 |
| 3 | 29477 |
| 4 | 24457 |
| 6 | 18186 |
| 8 | 13886 |
| 10 | 11193 |
| 12 | 9135 |

As those skilled in the art would readily appreciate, reducing or increasing the number of light sources (e.g., UV bulbs) or varying the light sources' wattage can impact the amount of UV radiation dosage supplied by the apparatus. Moreover, as discussed above, the amount of UV radiation dosage required to adequately inactive a microorganism (e.g., disinfect a surface), depends on the type of microorganism at target.

In some embodiments, the disinfection process may include moving the light-source housing assembly over the disinfection zone in a continuous manner. For example, sweeping the light-source housing assembly over a floor surface. In such cases, it may be desirable for the apparatus to provide a greater UV radiation dosage since the light-source housing assembly may only be positioned above a disinfection zone for a short duration of time (e.g., 1-2 seconds).

To aid in in a disinfection process, such as a disinfection process utilizing the method 1100, the apparatus may include one or more sensors. In some embodiments, the sensors may be in communication with a computing device, such as the computing device described in greater detail with FIG. 12. In such cases, the sensors may be used to determine a disinfection position for positioning the light-source housing assembly with respect to a disinfection zone. The disinfection position may include a distance from the disinfection zone that the light-source housing assembly should be to achieve adequate disinfection (e.g., for administering an adequate UV radiation dosage for disinfection). For example, the sensors may provide feedback, such as an alert (e.g., audible, visible, or haptic) that informs a user that the light-source housing assembly is positioned at a disinfection position. The sensors, in combination with the computing device, may also alert a user that a disinfection process is completed. For example, an alarm may be provided to indicate that the disinfection zone has received adequate UV radiation, or the sensors may determine that an adequate amount of UV radiation has been dispensed onto the disinfection zone.

Referring now to FIG. 12, an example computing device 1200 suitable for use in example systems or methods for distribution of UV radiation, according to an embodiment herein, is shown. The example computing device 1200 may be used in combination with any apparatus provided herein (e.g., the apparatus 100, 200, 300, etc.) The computing device 1200 may be local or may be remote from the apparatus. For example, the computing device 1200 may be a handheld device, such as a cell phone or tablet, that is paired with the apparatus for providing distribution of UV radiation. In other embodiments, the computing device 1200 may be part of the apparatus, such as a display on the apparatus for providing information and feedback on a disinfection process.

The example computing device 1200 includes a processor 1210 which is in communication with the memory 1220 and other components of the computing device 1200 using one or more communications buses 1202. The processor 1210 is configured to execute processor-executable instructions stored in the memory 1220 to perform one or more methods for distribution UV radiation, such as part or all of the example method 1100, described above with respect to FIG. 11. The computing device, in this example, also includes one or more user input devices, such as a keyboard, mouse, touchscreen, video input device (e.g., one or more cameras), microphone, etc., to accept user input. The computing device 1200 may, in some embodiments, include a display 1240 to provide visual output to a user.

The computing device 1200 also includes a communications interface 1230. In some examples, the communications interface 1230 may enable communications using one or more networks, including a local area network (“LAN”); wide area network (“WAN”), such as the Internet; metropolitan area network (“MAN”); point-to-point or peer-to-peer connection; etc. Communication with other devices may be accomplished using any suitable networking protocol. For example, one suitable networking protocol may include the Internet Protocol (“IP”), Transmission Control Protocol (“TCP”), User Datagram Protocol (“UDP”), or combinations thereof, such as TCP/IP or UDP/IP.

The computing device 1200 may be configured to execute one or more instructions, such as software 1260. The software 1260 may be remotely or locally executed by the computing device 1200. In some embodiments, the software 1260 may include instructions, which when executed by the processor 1210, cause the apparatus to perform one or more steps of the method 1100. For example, instructions may allow for one or more steps of the method 1100 to be performed automatically.

In some embodiments, the computing device 1200 may be in operable communication with one or more sensors of the apparatus. For example, the computing device 1200 may be in operable communication with the sensors 250 of the apparatus 200. The sensors of the apparatus may provide the computing device 1200 with positional data of a respective light-source housing assembly. This can allow the computing device 1200, using the software 1260, to determine if the light-source housing assembly is in a correct position with respect to a disinfection zone. For example, the computing device 1200 may provide a user with an indication, such as an audible alert, a haptic alert, or visual alert when the light-source housing assembly is within a distance of the disinfection zone to adequately disinfect the disinfection zone. In other embodiments, the computing device 1200 may provide a user with a timing alert (e.g., audible, visible, or haptic) to notify the user of an adequate time for positioning the light-source housing assembly over the disinfection zone. For example, the computing device 1200 may provide a timer. The timer may indicate to the user how long to hold the light-source housing assembly over a first disinfection zone to achieve adequate disinfection. Once the timer is completed, the user may be alerted that disinfection of the first disinfection zone is complete and to move the light-source housing assembly to a second disinfection zone.

In some embodiments, the computing device 1200 may be in operable communication with one or more environmental sensors. For example, one or more environmental sensors may be within the disinfection area. The environmental sensors may determine an output of UV radiation from the apparatus and provide feedback to the computing device 1200 as to whether an adequate amount of UV radiation is applied to the disinfection zone.

In some embodiments, the computing device 1200 may be configured to execute instructions to automatically position one or more of the arms or light-source housing assemblies of a device. For example, as described above with respect to FIG. 11, the software 1260 may include instructions for determining a disinfection zone, modifying a position of a light-source housing assembly based on the disinfection zone, and disinfecting the disinfection zone based on the position of the light-source housing assembly. Based on these instructions, the computer device 1200 may transmit instructions to the apparatus to move a respective component such to position the light-source housing assembly in an adequate position with respect to the disinfection zone.

While some examples of methods and systems herein are described in terms of software executing on various machines, the methods and systems may also be implemented as specifically-configured hardware, such as field-programmable gate array (FPGA) specifically to execute the various methods according to this disclosure. For example, examples can be implemented in digital electronic circuitry, or in computer hardware, firmware, software, or in a combination thereof. In one example, a device may include a processor or processors. The processor comprises a computer-readable medium, such as a random access memory (RAM) coupled to the processor. The processor executes computer-executable program instructions stored in memory, such as executing one or more computer programs. Such processors may comprise a microprocessor, a digital signal processor (DSP), an application-specific integrated circuit (ASIC), field programmable gate arrays (FPGAs), and state machines. Such processors may further comprise programmable electronic devices such as PLCs, programmable interrupt controllers (PICs), programmable logic devices (PLDs), programmable read-only memories (PROMs), electronically programmable read-only memories (EPROMs or EEPROMs), or other similar devices.

Such processors may comprise, or may be in communication with, media, for example one or more non-transitory computer-readable media, that may store processor-executable instructions that, when executed by the processor, can cause the processor to perform methods according to this disclosure as carried out, or assisted, by a processor. Examples of non-transitory computer-readable medium may include, but are not limited to, an electronic, optical, magnetic, or other storage device capable of providing a processor, such as the processor in a web server, with processor-executable instructions. Other examples of non-transitory computer-readable media include, but are not limited to, a floppy disk, CD-ROM, magnetic disk, memory chip, ROM, RAM, ASIC, configured processor, all optical media, all magnetic tape or other magnetic media, or any other medium from which a computer processor can read. The processor, and the processing, described may be in one or more structures, and may be dispersed through one or more structures. The processor may comprise code to carry out methods (or parts of methods) according to this disclosure.

The foregoing description of some examples has been presented only for the purpose of illustration and description and is not intended to be exhaustive or to limit the disclosure to the precise forms disclosed. Numerous modifications and adaptations thereof will be apparent to those skilled in the art without departing from the spirit and scope of the disclosure.

Reference herein to an example or implementation means that a particular feature, structure, operation, or other characteristic described in connection with the example may be included in at least one implementation of the disclosure. The disclosure is not restricted to the particular examples or implementations described as such. The appearance of the phrases “in one example,” “in an example,” “in one implementation,” or “in an implementation,” or variations of the same in various places in the specification does not necessarily refer to the same example or implementation. Any particular feature, structure, operation, or other characteristic described in this specification in relation to one example or implementation may be combined with other features, structures, operations, or other characteristics described in respect of any other example or implementation.

Use herein of the word “or” is intended to cover inclusive and exclusive OR conditions. In other words, A or B or C includes any or all of the following alternative combinations as appropriate for a particular usage: A alone; B alone; C alone; A and B only; A and C only; B and C only; and A and B and C.

### EXAMPLES

These illustrative examples are mentioned not to limit or define the scope of this disclosure, but rather to provide examples to aid understanding thereof. Illustrative examples are discussed above in the Detailed Description, which provides further description. Advantages offered by various examples may be further understood by examining this specification.

As used below, any reference to a series of examples is to be understood as a reference to each of those examples disjunctively (e.g., “Examples 1-4” is to be understood as “Examples 1, 2, 3, or 4”).

Example 1 is an apparatus for distribution ultraviolet (UV) radiation, the apparatus comprising: a light-source housing assembly comprising a first end and a second end, wherein the light-source housing assembly comprises: a first light-source holder configured to hold a first end of at least one UV bulb within the light-source housing assembly, wherein the first light-source holder is positioned proximate to the first end of the light-source housing assembly; a second light-source holder configured to hold a second end of the at least one UV bulb within the light-source housing assembly, wherein the second light-source holder is positioned proximate to the second end of the light-source housing assembly, wherein the at least one UV bulb is optionally included; a directionalization cover extending from the first end of the light-source housing assembly to the second end of the light-source housing assembly, wherein the directionalization cover comprises at least one surface for directing emittance of UV light from the at least one UV bulb; and a transmittance cover extending from the first end of the light-source housing assembly to the second end of the light-source housing assembly, wherein the transmittance cover transmits the UV light from the at least one UV bulb; a power source configured to provide power to the at least one UV bulb of the light-source housing assembly; an arm comprising a distal end and a proximal end, wherein the arm comprises a moveable element; and an attachment component, wherein the attachment component is moveably coupled to the proximal end of the arm and moveably coupled to the first end of the light-source housing assembly.

Example 2 is the apparatus of any previous or subsequent Example, wherein the apparatus comprises the at least one UV bulb held by the first light-source holder and the second light-source holder in the light-source housing assembly.

Example 3 is the apparatus of any previous or subsequent Example, wherein the arm comprises a first segment and a second segment, wherein the moveable element forms a telescoping arm with the first segment and the second segment.

Example 4 is the apparatus of any previous or subsequent Example, wherein the attachment component is moveably coupled to the light-source housing assembly by a pivoting component allowing for at least 180° rotation of the light-source housing assembly with respect to a longitudinal axis of the arm.

Example 5 is the apparatus of any previous or subsequent Example, wherein the pivoting component allows for 360° rotation of the light-source housing assembly with respect to the longitudinal axis of the arm.

Example 6 is the apparatus of any previous or subsequent Example, wherein the attachment component provides 6 degrees of freedom for the light-source housing assembly with respect to the arm.

Example 7 is the apparatus of any previous or subsequent Example, wherein the arm comprises a first segment and a second segment, wherein the second segment is: coupled to the attachment component at the proximal end of the arm; and independently rotatable from the first segment.

Example 8 is the apparatus of any previous or subsequent Example, wherein the second segment is rotatably independent from the first segment such that the first segment rotates in a first direction that is transverse to a longitudinal axis of the second segment.

Example 9 is the apparatus of any previous or subsequent Example, wherein the attachment component further comprises: a pivotable connection; and a stub arm comprising a first end and a second end, wherein: the pivotable connection couples the second end of the stub arm to the proximal end of the arm; and the first end of the stub arm is coupled to the first end of the light-source housing assembly, wherein the pivotable connection allows the stub arm to be rotatably independent with respect to the arm, allowing for at least 180° rotation of the stub arm with respect to the arm.

Example 10 is the apparatus of any previous or subsequent Example, wherein the at least one surface of the directionalization cover comprises a first surface, a second surface, and a third surface, wherein the first surface is oriented to direct emittance of UV light in a first direction, the second surface is oriented to direct emittance of the UV light in a second direction, and the third surface is oriented to direct emittance of the UV light in a third direction, wherein the first direction, second direction, and third direction are different directions from each other.

Example 11 is the apparatus of any previous or subsequent Example, wherein the power source comprises one or more of: a battery; an outlet plug; or a motorized vehicle.

Example 12 is the apparatus of any previous or subsequent Example, wherein the at least one UV bulb comprises two UV bulbs, and wherein: the first light-source holder is configured to hold the first end of a first UV bulb and a first end of a second UV bulb; and the second light-source holder is configured to hold the second end of the first UV bulb and a second end of the second UV bulb.

Example 13 is the apparatus of any previous or subsequent Example, wherein the apparatus is a handheld device.

Example 14 is the apparatus of any previous or subsequent Example, the apparatus further comprising: a second attachment component, wherein the second attachment component is coupled to the distal end of the arm and is configured to couple the arm with a motorized vehicle.

Example 15 is the apparatus of any previous or subsequent Example, wherein the second attachment component removably couples the distal end of the arm with the motorized vehicle.

Example 16 is the apparatus of any previous or subsequent Example, the apparatus further comprising a computing system, and wherein the light-source housing assembly comprises one or more sensors for providing positional feedback on a position of the light-source housing assembly to the computing system.

Example 17 is the apparatus of any previous or subsequent Example, wherein the one or more sensors are further configured to provide feedback of a disinfection zone achieved by the light-source housing assembly to the computer system.

Example 18 is the apparatus of any previous or subsequent Example, the apparatus further comprising: a second light-source housing assembly comprising a first end and a second end, wherein the second light-source housing assembly comprises: a third light-source holder configured to hold a first end of at least one UV bulb within the second light-source housing assembly; a fourth light-source holder configured to hold a second end of the at least one UV bulb within the second light-source housing assembly; and a second directionalization cover extending from the first end of the second light-source housing assembly to the second end of the second light-source housing assembly, wherein the directionalization cover comprises at least one surface for directing emittance of UV light from the at least one UV bulb; and a second transmittance cover extending from the first end of the second light-source housing assembly to the second end of the second light-source housing assembly, wherein the transmittance cover transmits the UV light from the at least one UV bulb, wherein the power source is further configured to provide power to the at least one UV bulb of the second light-source housing assembly.

Example 19 is the apparatus of any previous or subsequent Example, the apparatus further comprising: a pivotable connection; a first stub arm comprising a first end and a second end, wherein: the pivotable connection couples the second end of the first stub arm to the proximal end of the arm; and the first end of the first stub arm is coupled to the first end of the light-source housing assembly; and a second stub arm comprising a first end and a second end, wherein: pivotable connection couples the second end of the second stub arm to the proximal end of the arm; and the first end of the second stub arm is coupled to the first end of the second light-source housing assembly, wherein the pivotable connection allows the first stub arm to be rotatably independent with respect to the arm and the second stub arm to be rotatably independent with respect to the arm.

Example 20 is a method for disinfecting a surface using UV radiation, the method comprising: providing an apparatus for distributing UV radiation, wherein the apparatus comprises: a light-source housing assembly comprising a first end and a second end, wherein the light-source housing assembly comprises: at least one UV bulb operable to emit UV radiation; a first light-source holder configured to hold a first end of the at least one UV bulb within the light-source housing assembly, wherein the first light-source holder is positioned proximate to the first end of the light-source housing assembly; a second light-source holder configured to hold a second end of the at least one UV bulb within the light-source housing assembly, wherein the second light-source holder is positioned proximate to the second end of the light-source housing assembly; a directionalization cover extending from the first end of the light-source housing assembly to the second end of the light-source housing assembly, wherein the directionalization cover comprises at least one surface for directing emittance of UV light from the at least one UV bulb; and a transmittance cover extending from the first end of the light-source housing assembly to the second end of the light-source housing assembly, wherein the transmittance cover transmits the UV light from the at least one UV bulb; a power source configured to provide power to the at least one UV bulb of the light-source housing assembly; an arm comprising a distal end and a proximal end, wherein the arm comprises a moveable element; and an attachment component, wherein the attachment component is moveably coupled to the proximal end of the arm and moveably coupled to the first end of the light-source housing assembly; positioning the light-source housing assembly in a first position relative to the arm; distributing UV radiation using the light-source housing assembly in the first position; positioning the light-source housing assembly in a second position relative to the arm; and distributing UV radiation using the light-source housing assembly in the second position.

Example 21 is the method of any previous or subsequent Example, wherein: positioning the light-source housing assembly in the first position relative to the arm comprises directionalizing the UV radiation from the at least one UV bulb towards a first disinfection zone; and positioning the light-source assembly in the second position relative to the arm comprises directionalizing the UV radiation from the at least one UV bulb towards a second disinfection zone, wherein the first disinfection zone is different from the second disinfection zone.

Example 22 is the method of any previous or subsequent Example, wherein distributing UV radiation using the light-source assembly in the first position comprises providing a UV radiation dosage that is greater than 10,000 μW/cm² at a distance of 10 inches or less from a disinfection zone.

1. An apparatus for distribution ultraviolet (UV) radiation, the apparatus comprising: a light-source housing assembly comprising a first end and a second end, wherein the light-source housing assembly comprises:
a first light-source holder configured to hold a first end of at least one UV bulb within the light-source housing assembly, wherein the first light-source holder is positioned proximate to the first end of the light-source housing assembly;
a second light-source holder configured to hold a second end of the at least one UV bulb within the light-source housing assembly, wherein the second light-source holder is positioned proximate to the second end of the light-source housing assembly, wherein the at least one UV bulb is optionally included;
a directionalization cover extending from the first end of the light-source housing assembly to the second end of the light-source housing assembly, wherein the directionalization cover comprises at least one surface for directing emittance of UV light from the at least one UV bulb; and
a transmittance cover extending from the first end of the light-source housing assembly to the second end of the light-source housing assembly, wherein the transmittance cover transmits the UV light from the at least one UV bulb;
a power source configured to provide power to the at least one UV bulb of the lightsource housing assembly;
an arm comprising a distal end and a proximal end, wherein the arm comprises a moveable element; and
an attachment component, wherein the attachment component is moveably coupled to the proximal end of the arm and moveably coupled to the first end of the light-source housing assembly.

2. The apparatus of claim 1, wherein the apparatus comprises the at least one UV bulb held by the first light-source holder and the second light-source holder in the light-source housing assembly.

3. The apparatus of claim 1, wherein the arm comprises a first segment and a second segment, wherein the moveable element forms a telescoping arm with the first segment and the second segment.

4. The apparatus of claim 1, wherein the attachment component is moveably coupled to the light-source housing assembly by a pivoting component allowing for at least 180° rotation of the light-source housing assembly with respect to a longitudinal axis of the arm.

5. The apparatus of claim 4, wherein the pivoting component allows for 360° rotation of the light-source housing assembly with respect to the longitudinal axis of the arm.

6. The apparatus of claim 1, wherein the attachment component provides 6 degrees of freedom for the light-source housing assembly with respect to the arm.

7. The apparatus of claim 1, wherein the arm comprises a first segment and a second segment, wherein the second segment is:
coupled to the attachment component at the proximal end of the arm; and independently rotatable from the first segment.

8. The apparatus of claim 7, wherein the second segment is rotatably independent from the first segment such that the first segment rotates in a first direction that is transverse to a longitudinal axis of the second segment.

9. The apparatus of claim 1, wherein the attachment component further comprises:
a pivotable connection; and
a stub arm comprising a first end and a second end, wherein:
the pivotable connection couples the second end of the stub arm to the proximal end of the arm; and
the first end of the stub arm is coupled to the first end of the light-source housing assembly,
wherein the pivotable connection allows the stub arm to be rotatably independent with respect to the arm, allowing for at least 180° rotation of the stub arm with respect to the arm.

10. The apparatus of claim 1, wherein the at least one surface of the directionalization cover comprises a first surface, a second surface, and a third surface, wherein the first surface is oriented to direct emittance of UV light in a first direction, the second surface is oriented to direct emittance of the UV light in a second direction, and the third surface is oriented to direct emittance of the UV light in a third direction, wherein the first direction, second direction, and third direction are different directions from each other.

11. (canceled)

12. The apparatus of a claim 1, wherein the at least one UV bulb comprises two UV bulbs, and wherein:
the first light-source holder is configured to hold the first end of a first UV bulb and a first end of a second UV bulb; and
the second light-source holder is configured to hold the second end of the first UV bulb and a second end of the second UV bulb.

13. (canceled)

14. The apparatus of claim 1, the apparatus further comprising:
a second attachment component, wherein the second attachment component is coupled to the distal end of the arm and is configured to couple the arm with a motorized vehicle.

15. The apparatus of claim 14, wherein the second attachment component removably couples the distal end of the arm with the motorized vehicle.

16. The apparatus of claim 1, the apparatus further comprising a computing system, and wherein the light-source housing assembly comprises one or more sensors for providing positional feedback on a position of the light-source housing assembly to the computing system.

17. The apparatus of claim 16, wherein the one or more sensors are further configured to provide feedback of a disinfection zone achieved by the light-source housing assembly to the computing system.

18. The apparatus of claim 1, the apparatus further comprising:
a second light-source housing assembly comprising a first end and a second end, wherein the second light-source housing assembly comprises:
a third light-source holder configured to hold a first end of at least one UV bulb within the second light-source housing assembly;
a fourth light-source holder configured to hold a second end of the at least one UV bulb within the second light-source housing assembly; and
a second directionalization cover extending from the first end of the second lightsource housing assembly to the second end of the second light-source housing assembly, wherein the directionalization cover comprises at least one surface for directing emittance of UV light from the at least one UV bulb; and
a second transmittance cover extending from the first end of the second lightsource housing assembly to the second end of the second light-source housing assembly, wherein the transmittance cover transmits the UV light from the at least one UV bulb, wherein the power source is further configured to provide power to the at least one UV bulb of the second light-source housing assembly.

19. The apparatus of claim 18, the apparatus further comprising:
a pivotable connection;
a first stub arm comprising a first end and a second end, wherein:
the pivotable connection couples the second end of the first stub aim to the proximal end of the arm; and
the first end of the first stub arm is coupled to the first end of the light-source housing assembly; and
a second stub arm comprising a first end and a second end, wherein:
pivotable connection couples the second end of the second stub arm to the proximal end of the arm; and
the first end of the second stub arm is coupled to the first end of the second lightsource housing assembly,
wherein the pivotable connection allows the first stub arm to be rotatably independent with respect to the arm and the second stub arm to be rotatably independent with respect to the arm.

20. A method for disinfecting a surface using UV radiation, the method comprising: providing an apparatus for distributing UV radiation, wherein the apparatus comprises:
a light-source housing assembly comprising a first end and a second end, wherein the light-source housing assembly comprises:
at least one UV bulb operable to emit UV radiation;
a first light-source holder configured to hold a first end of the at least one UV bulb within the light-source housing assembly, wherein the first light-source holder is positioned proximate to the first end of the light-source housing assembly;
a second light-source holder configured to hold a second end of the at least one UV bulb within the light-source housing assembly, wherein the second lightsource holder is positioned proximate to the second end of the light-source housing assembly;
a directionalization cover extending from the first end of the light-source housing assembly to the second end of the light-source housing assembly, wherein the directionalization cover comprises at least one surface for directing emittance of UV light from the at least one UV bulb; and
a transmittance cover extending from the first end of the light-source housing assembly to the second end of the light-source housing assembly, wherein the transmittance cover transmits the UV light from the at least one UV bulb;
a power source configured to provide power to the at least one UV bulb of the light-source housing assembly;
an aim comprising a distal end and a proximal end, wherein the arm comprises a moveable element; and
an attachment component, wherein the attachment component is moveably coupled to the proximal end of the arm and moveably coupled to the first end of the lightsource housing assembly;
positioning the light-source housing assembly in a first position relative to the arm;
distributing UV radiation using the light-source housing assembly in the first position;
positioning the light-source housing assembly in a second position relative to the arm; and
distributing UV radiation using the light-source housing assembly in the second position.

21. The method of claim 20, wherein:
positioning the light-source housing assembly in the first position relative to the arm comprises directionalizing the UV radiation from the at least one UV bulb towards a first disinfection zone; and
positioning the light-source assembly in the second position relative to the arm comprises directionalizing the UV radiation from the at least one UV bulb towards a second disinfection zone,
wherein the first disinfection zone is different from the second disinfection zone.

22. The method of claim 20, wherein distributing UV radiation using the lightsource assembly in the first position comprises providing a UV radiation dosage that is greater than 10,000 pW/cm² at a distance of 10 inches or less from a disinfection zone.
